(19) United States
(12) Patent Application Publication
(10) Pub. No.: US 2023/0050043 A1
(43) Pub. Date: Feb. 16, 2023

(54) Compositions Comprising Microbes and Methods of Use and Making Thereof

(71) Applicant: Pendulum Therapeutics, Inc., San Francisco, CA (US)

(72) Inventors: Jeewon Garcia-So, Indio, CA (US); Nicholas B. Justice, SE Atlanta, GA (US); Magdalena K. Stoeva, Oakland, CA (US); John S. Eid, San Francisco, CA (US); Julia N. Myers, San Francisco, CA (US); Andrew T. Cheng, San Francisco, CA (US); Moe Brandi, San Francisco, CA (US); Paul McMurdie, San Francisco, CA (US); James H. Bullard, San Francisco, CA (US)

(21) Appl. No.: 17/787,880

(22) PCT Filed: Dec. 22, 2020
(86) PCT No.: PCT/US2020/066634

§ 371 Date: Jun. 21, 2022

Related U.S. Application Data
(60) Provisional application No. 62/953,005, filed on Dec. 23, 2019.

(51) Int. Cl. A61K 35/741; A61K 31/733; A61K 35/745; A61K 35/744; A61P 1/00; A61K 35/742; A23L 33/135; A23L 33/21

(52) U.S. Cl. CPC A61K 35/741; A61K 31/733; A61K 35/745; A61K 35/744; A61P 1/00; A61K 35/742; A23L 33/135; A23L 33/21; A23V 2002/00; A23Y 2300/19

(57) ABSTRACT

The disclosure provides microbial compositions (e.g., compositions comprising one or more microbes or protein components thereof), that can be administered to subjects to confer beneficial effects. Disclosed compositions can, for example, be useful in reducing visceral hypersensitivity, or pain in response to colorectal distension.

### CROSS REFERENCE

This application claims priority to U.S. Provisional Patent Application No. 62/953,005, filed Dec. 23, 2019, which is incorporated herein by reference in its entirety.

### BACKGROUND

The body of an individual is inhabited by trillions of microbes across various locations, often referred to as microbiomes. Microbiomes can play a key role in many health conditions and diseases. Despite the interrelation between microbiomes and health, the complexity of the various microbiomes, as well as difficulties in characterizing, categorizing, and analyzing microbiome constituents has made understanding microbiomes challenging. Consequently, these challenges have presented hurdles in the development of diagnostic and therapeutic applications for microbiome-related health conditions and diseases. The present disclosure provides methods, systems, compositions, and kits to address the need for microbiome-related treatment of health conditions and disease.

### SUMMARY

In embodiments, disclosed herein are compositions comprising one or more microbes selected from the group consisting of Akkermansia sp., Anaerostipes sp., Bacteroides sp., Bifidobacterium sp., Blautia sp., Clostridium sp., Collinsella sp., Coprococcus sp., Eubacterium sp., and Ruminococcus sp.

In embodiments, disclosed herein are compositions comprising microbes from 2 or more, 3 or more, four or more, five or more, six or more, seven or more, eight or more, nine or more, or all ten of the group consisting of Akkermansia sp., Anaerostipes sp., Bacteroides sp., Bifidobacterium sp., Blautia sp., Clostridium sp., Collinsella sp., Coprococcus sp., Eubacterium sp., and Ruminococcus sp.

In embodiments, disclosed herein are compositions comprising microbes selected from 2 or more, 3 or more, four or more, or all five of the group consisting of Bifidobacterium sp., Ruminococcus sp., Blautia sp., Anaerostipes sp., and Coprococcus sp.

In embodiments, disclosed herein are compositions comprising microbes are selected from 2 or more, 3 or more, or all four of the group consisting of Bifidobacterium sp., Akkermansia sp., Clostridium sp., and Eubacterium sp.

In embodiments, disclosed herein are compositions comprising microbes are selected from 2 or more, 3 or more, four or more, five or more, six or more, or all seven of the group consisting of Eubacterium sp., Clostridium sp., Bifidobacterium sp., Collinsella sp., Bacteroides sp., Blautia sp., and Bacteroides sp.

In embodiments, disclosed herein are compositions comprising one or more microbes having a 16S rRNA sequence comprising at least 95% identity to the full length of a 16S rRNA sequence of a microbe selected from the group consisting of Akkermansia mucimphila ATCC BAA-835, Anaerostipes caccae DSM 14662, Bacteroides finegoldii DSM 17565, Bacteroides ovatus ATCC 8483, Bacteroides stercoris ATCC 43183, Bifidobacterium adolescentis ATCC 15703, Bifidobacterium infantis ATCC 15697, Bifidobacterium faecale JCM 19861, Bifidobacterium longum ATCC 15697, Blautia hydrogenotrophica DSM 10507, Blautia producta ATCC 27340, Clostridium butyricum DSM 10702, Clostridium beijerinckii NCIMB 8052, Clostridium innocuum ATCC 14501, Clostridium sporogenes DSM 795, Collinsella aerofaciens ATCC 25986, Coprococcus comes ATCC 27758, Eubacterium hallii DSM 3353, Eubacterium limosum ATCC 5486, and Ruminococcus faecis JCM 15917.

In embodiments, disclosed herein are compositions comprising microbes having 16S rRNA sequence comprising at least 95% identity to the full length of a 16S rRNA sequence of a microbe from 2 or more, 3 or more, four or more, five or more, six or more, seven or more, eight or more, nine or more, ten or more, eleven or more, or all twelve of the group consisting of Eubacterium limosum ATCC 5486, Clostridium innocuum ATCC 14501, Bifidobacterium faecale JCM 19861, Collinsella aerofaciens ATCC 25986, Bacteroides stercoris ATCC 43183, Bifidobacterium adolescentis ATCC 15703, Bifidobacterium infantis ATCC 15697, Bifidobacterium longum ATCC 15697, Clostridium sporogenes DSM 795, Blautia hydrogenotrophica DSM 10507, Bacteroides ovatus ATCC 8483, and Bacteroides finegoldii DSM 17565.

In embodiments, disclosed herein are compositions comprising microbes having 16S rRNA sequence comprising at least 95% identity to the full length of a 16S rRNA sequence of a microbe from 2 or more, 3 or more, four or more, or all five of the group consisting of Bifidobacterium infantis ATCC 15697, Akkermansia mucimphila ATCC BAA-835, Clostridium butyricum DSM 10702, Eubacterium hallii DSM 3353, Clostridium beijerinckii NCIMB 8052.

In embodiments, disclosed herein are compositions comprising microbes having 16S rRNA sequence comprising at least 97% identity to the full length of a 16S rRNA sequence of a microbe from 2 or more, 3 or more, four or more, five or more, six or more, seven or more, eight or more, nine or more, ten or more, eleven or more, or all twelve of the group consisting of Eubacterium limosum ATCC 5486, Clostridium innocuum ATCC 14501, Bifidobacterium faecale JCM 19861, Collinsella aerofaciens ATCC 25986, Bacteroides stercoris ATCC 43183, Bifidobacterium adolescentis ATCC 15703, Bifidobacterium infantis ATCC 15697, Bifidobacterium longum ATCC 15697, Clostridium sporogenes DSM 795, Blautia hydrogenotrophica DSM 10507, Bacteroides ovatus ATCC 8483, and Bacteroides finegoldii DSM 17565.

In embodiments, disclosed herein are compositions comprising microbes having 16S rRNA sequence comprising at least 97% identity to the full length of a 16S rRNA sequence of a microbe from 2 or more, 3 or more, four or more, or all five of the group consisting of Anaerostipes caccae DSM 14662, Bifidobacterium adolescentis ATCC 15703, Blautia producta ATCC 27340, Coprococcus comes ATCC 27758, and Ruminococcus faecis JCM 15917.

In embodiments, disclosed herein are compositions comprising microbes having 16S rRNA sequence comprising at least 97% identity to the full length of a 16S rRNA sequence of a microbe from 2 or more, 3 or more, four or more, or all five of the group consisting of Anaerostipes caccae, Bifidobacterium adolescentis, Blautia producta, Coprococcus comes, and Ruminococcus faecis.

In embodiments, disclosed herein are compositions comprising microbes having 16S rRNA sequence comprising at least 97% identity to the full length of a 16S rRNA sequence of a microbe from 2 or more, 3 or more, four or more, or all five of the group consisting of Bifidobacterium infantis ATCC 15697, Akkermansia muciniphila ATCC BAA-835, Clostridium butyricum DSM 10702, Eubacterium hallii DSM 3353, Clostridium beijerinckii NCIMB 8052.

In embodiments, disclosed herein are compositions comprising microbes having 16S rRNA sequence comprising at least 95% identity to the full length of a 16S rRNA sequence of a microbe from 2 or more, 3 or more, four or more, or all five of the group consisting of Bifidobacterium adolescentis ATCC 15703, Ruminococcus faecis JCM 15917, Blautia producta ATCC 27340, Anaerostipes caccae DSM 14662, and Coprococcus comes ATCC 27758.

In embodiments, disclosed herein are compositions comprising one or more microbes selected from the group consisting of Akkermansia muciniphila, Anaerostipes caccae, Bacteroides finegoldii, Bacteroides ovatus, Bacteroides stercoris, Bifidobacterium adolescentis, Bifidobacterium infantis, Bifidobacterium faecale, Bifidobacterium longum, Blautia hydrogenotrophica, Blautia producta, Clostridium butyricum, Clostridium beijerinckii, Clostridium innocuum, Clostridium sporogenes, Collinsella aerofaciens, Coprococcus comes, Eubacterium hallii, Eubacterium limosum, and Ruminococcus faecis.

In embodiments, disclosed herein are compositions comprising Anaerostipes caccae, Bifidobacterium adolescentis, Blautia producta, Coprococcus comes, and Ruminococcus faecis.

In embodiments, disclosed herein are compositions comprising microbes from 2 or more, 3 or more, four or more, or all five of the group consisting of Anaerostipes caccae DSM 14662, Bifidobacterium adolescentis ATCC 15703, Blautia producta ATCC 27340, Coprococcus comes ATCC 27758, and Ruminococcus faecis JCM 15917.

In embodiments, disclosed herein are compositions comprising Anaerostipes caccae DSM 14662, Bifidobacterium adolescentis ATCC 15703, Blautia producta ATCC 27340, Coprococcus comes ATCC 27758, and Ruminococcus faecis JCM 15917.

In embodiments, disclosed herein are compositions comprising Anaerostipes caccae, and Coprococcus comes.

In embodiments, disclosed herein are compositions comprising Anaerostipes caccae, Blautia producta, and Coprococcus comes.

In embodiments, disclosed herein are compositions comprising Anaerostipes caccae DSM 14662, and Coprococcus comes ATCC 27758.

In embodiments, disclosed herein are compositions comprising Anaerostipes caccae DSM 14662, Blautia producta ATCC 27340, and Coprococcus comes ATCC 27758.

In embodiments, disclosed herein are compositions comprising microbes from 2 or more, 3 or more, four or more, or all five of the group consisting of Bifidobacterium infantis, Akkermansia muciniphila, Clostridium butyricum, Eubacterium hallii, Clostridium beijerinckii.

In embodiments, disclosed herein are compositions comprising microbes from 2 or more, 3 or more, four or more, five or more, six or more, seven or more, eight or more, nine or more, ten or more, eleven or more, or all twelve of the group consisting of Eubacterium limosum, Clostridium innocuum, Bifidobacterium faecale, Collinsella aerofaciens, Bacteroides stercoris, Bifidobacterium adolescentis, Bifidobacterium infantis, Bifidobacterium longum, Clostridium sporogenes, Blautia hydrogenotrophica, Bacteroides ovatus, Bacteroides finegoldii.

In embodiments, disclosed herein are compositions comprising microbes from 2 or more, 3 or more, four or more, or all five of the group consisting of Bifidobacterium adolescentis, Ruminococcus faecis, Blautia producta, Anaerostipes caccae, and Coprococcus comes.

Certain embodiments include a composition of any of the preceding embodiments, wherein the formulation further comprises one or more additional microbe strains having a 16S rRNA sequence comprising at least 95% identity to the full length of a 16S rRNA sequence of a microbe selected from the group consisting of Akkermansia muciniphila, Anaerostipes caccae, Bacteroides finegoldii, Bacteroides ovatus, Bacteroides stercoris, Eubactrium hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium butyricum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof.

Certain embodiments include a composition of any of the preceding embodiments, wherein the formulation further comprises one or more additional microbe strains having a 16S rRNA sequence comprising at least 97% identity to the full length of a 16S rRNA sequence of a microbe selected from the group consisting of Akkermansia muciniphila, Anaerostipes caccae, Bacteroides finegoldii, Bacteroides ovatus, Bacteroides stercoris, Eubactrium hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium butyricum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof.

Certain embodiments include a composition or a composition of any of the preceding embodiments comprising at least one effective protein component extracted from at least one of the microbes selected from the group consisting of Akkermansia mucimphila, Anaerostipes caccae, Bacteroides finegoldii, Bacteroides ovatus, Bacteroides stercoris, Eubactrium hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium butyricum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof.

A composition comprising a first group of one or more microbes that produces an intermediate molecule from a prebiotic, wherein the intermediate molecule is any one or more of acetate, lactate or glucose and a second group of one or more microbes that uses the intermediate molecule to produce butyrate.

Certain embodiments include a composition of any of the preceding embodiments, wherein the formulation reduces visceral motor reflex in the colon of a subject treated with the formulation.

Certain embodiments include a composition of any of the preceding embodiments, wherein the formulation reduces pain in response to colorectal distension in a subject treated with the composition.

Certain embodiments include a composition of any of the preceding embodiments, wherein the subject suffers from irritable bowel syndrome, inflammatory bowel disease, ulcerative colitis, diarrhea, constipation, leaky intestine, and/or Crohn's disease.

Certain embodiments include a composition of any of the preceding embodiments, wherein the subject is a mammal. In particular embodiments, the mammal is a human.

Certain embodiments include a composition of any of the preceding embodiments, wherein the formulation further comprises an enteric coating.

Certain embodiments include a composition of any of the preceding embodiments, wherein the composition is formulated as an enteric-coated pill. In some aspects, the method may comprise formulating the composition as an enteric-coated pill, wherein the enteric-coating is formed by a pH sensitive polymer. In some aspects, the method may comprise formulating the composition as an enteric-coated pill, wherein the enteric-coating is formed by a pH sensitive polymer, wherein the polymer is eudragit FS30D.

Certain embodiments include a composition of any of the preceding embodiments, wherein the formulation further comprises an effective amount of a preservative.

Certain embodiments include a composition of any of the preceding embodiments, wherein the formulation further comprises a prebiotic.

Certain embodiments include a composition of any of the preceding embodiments, wherein the formulation further comprises an enteric coating.

Certain embodiments include a composition of any of the preceding embodiments, wherein the formulation further comprises a prebiotic selected from the group consisting of inulin, green banana, reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof.

Certain embodiments include a composition of any of the preceding embodiments, wherein at least one of the microbes is lyophilized.

Certain embodiments include a composition of any of the preceding embodiments, wherein at least one of the microbes is viable.

Certain embodiments include a composition of any of the preceding embodiments, wherein at least one of the microbes is non-viable.

Certain embodiments include a composition of any of the preceding embodiments, wherein at least one of the microbes has been pasteurized.

Certain embodiments include a composition of any of the preceding embodiments, wherein the at least about 95% sequence identity is selected from the group consisting of: at least about 96%, at least about 97%, at least about 98%, at least about 99%, at least about 99.5%, and at least about 99.5% sequence identity to a rRNA sequence.

Certain embodiments include a composition of any of the preceding embodiments, wherein the pharmaceutical composition is substantially free of fecal matter obtained from a subject.

Certain embodiments include a composition of any of the preceding embodiments, wherein the at least one of the microbes comprises a population of the microbes.

Certain embodiments include a composition of any of the preceding embodiments, wherein the composition is formulated for oral delivery.

Certain embodiments include a composition of any of the preceding embodiments, wherein the composition is formulated as a nutritional supplement.

Certain embodiments include a composition of any of the preceding embodiments, wherein the composition is formulated as a medical food.

Certain embodiments include a composition of any of the preceding embodiments, wherein the composition is formulated as a medical probiotic.

Certain embodiments include a composition of any of the preceding embodiments, wherein the composition is formulated for dietary management of a gut disorder.

Certain embodiments include a composition of any of the preceding embodiments, wherein the composition is formulated for dietary management of Inflammatory bowel syndrome (IBS).

Certain embodiments include a composition of any of the preceding embodiments, wherein the composition is formulated for anal delivery.

Certain embodiments include a composition of any of the preceding embodiments, wherein the composition is formulated as a pill.

Certain embodiments include a composition of any of the preceding embodiments, wherein the composition is formulated as a capsule.

Certain embodiments include a composition of any of the preceding embodiments, wherein the composition is formulated in a liquid form suitable for administration via an enema.

Certain embodiments include a composition of any of the preceding embodiments, wherein the composition is formulated as a suppository.

Certain embodiments include a composition of any of the preceding embodiments, wherein the composition is formulated in a liquid form suitable for delivery via injection.

A method of producing the microbes of any of the preceding embodiments, the method comprising genetically-modifying the microbes to generate recombinant microbes. In some aspects, the method may comprise genetically-modifying the microbes to generate recombinant microbes, wherein an operon controls growth of the recombinant microbe.

Certain embodiments include a composition of any of the preceding embodiments, wherein the composition is formulated for delivery of the microbes to the subject's ileum region.

Certain embodiments include a composition of any of the preceding embodiments, wherein the composition is formulated for delivery of the microbes to the subject's colon region.

Certain embodiments include a composition of any of the preceding embodiments, wherein the composition is formulated for delivery of the microbes to the subject's ileum and colon region.

Certain embodiments include a composition of any of the preceding embodiments, wherein the microbes comprise a synergistic stability in the composition as compared to individual strains.

In some embodiments, disclosed herein is a method of treating a subject with at least one of the compositions of any of the preceding embodiments.

In some embodiments, disclosed herein is a method of reducing the visceral motor reflex in the colon of a subject comprising administering to the subject at least one of the compositions of any of the preceding embodiments.

In some embodiments, disclosed herein is a method of reducing pain in response to colorectal distension in a subject comprising administering to the subject at least one of the compositions of any of the preceding embodiments.

In particular embodiments, the subject suffers from irritable bowel syndrome, inflammatory bowel disease, ulcerative colitis, diarrhea, constipation, leaky intestine, and/or Crohn's disease.

Embodiments include the methods of any of the preceding embodiments, wherein treating and/or administering results in a subject with an altered microbiome.

Embodiments include the methods of the preceding embodiments, wherein treating and/or administering results in a subject with an altered gut microbiome.

Embodiments include the methods of the preceding embodiments, wherein the composition is co-administered with an antibiotic.

Embodiments include the methods of the preceding embodiments, wherein the composition is administered after an antibiotic. In some aspects, the method may comprise administering the composition after an antibiotic, wherein the composition is administered at least one hour after an antibiotic. In some aspects, the method may comprise administering the composition after an antibiotic, wherein the composition is administered at least 2 hours after an antibiotic. In some aspects, the method may comprise administering the composition after an antibiotic, wherein the composition is administered at least 12 hours after an antibiotic. In some aspects, the method may comprise administering the composition after an antibiotic, wherein the composition is administered at least 1 day after an antibiotic. In some aspects, the method may comprise administering the composition after an antibiotic, wherein the composition is administered at least 1 week after an antibiotic. In some aspects, the method may comprise administering the composition after an antibiotic, wherein the composition is administered at least 2 weeks after an antibiotic.

Embodiments include the methods of the preceding embodiments, wherein the composition is administered after completion of an antibiotic regimen by the subject.

Embodiments include the methods of the preceding embodiments, wherein the composition is formulated as a dietary supplement.

Embodiments include the methods of the preceding embodiments, wherein the composition is formulated as a nutritional supplement.

Embodiments include the methods of the preceding embodiments, wherein the composition is formulated as a medical food.

Embodiments include the methods of the preceding embodiments, wherein the composition is formulated as a medical probiotic.

Embodiments include the methods of the preceding embodiments, wherein the composition is a biologic product.

Embodiments include the methods of the preceding embodiments, further comprising determining the sequence of a population of the subject's microbiome by sequencing. In some aspects, the method may further comprise determining the sequence of the subject's microbiome by sequencing, the sequencing comprises sequencing the 16S rRNA. In some aspects, the method may further comprise determining the sequence of the subject's microbiome by sequencing, the sequencing comprises sequencing the 23S rRNA. In some aspects, the method may further comprise determining the sequence of the subject's microbiome by sequencing, the sequencing comprises sequencing the 23S and 16S rRNA. In some aspects, the method may further comprise determining the sequence of the subject's microbiome by sequencing, the sequencing comprises Complete Biome Test resolution. In some aspects, the sequencing comprises long-read sequencing. In some aspects, the method may further comprise determining the sequence of the subject's microbiome by sequencing, wherein the determining the sequence of the population of the subject's microbiome is performed before treating the subject with the composition. In some aspects, the method may further comprise determining the sequence of the subject's microbiome by sequencing, wherein the determining the sequence of the population of the subject's microbiome is performed after treating the subject with the composition.

Embodiments include the methods of the preceding embodiments, further comprising transmitting data via machine-readable code.

Embodiments include the methods of the preceding embodiments, further comprising computing data via machine-readable code.

Embodiments include the methods of the preceding embodiments, further comprising storing data via machine-readable code.

Embodiments include the methods of the preceding embodiments, wherein the method further comprises a companion diagnostic.

Embodiments include the methods of the preceding embodiments, wherein the composition is delivered to the subject's ileum region.

Embodiments include the methods of the preceding embodiments, wherein the composition is delivered to the subject's colon region.

Embodiments include the methods of the preceding embodiments, wherein the composition is delivered to the subject's ileum and colon region.

Embodiments include the methods of the preceding embodiments, wherein the composition is administered before food intake. In some aspects, the method may comprise administering the composition before food intake, wherein the composition is administered at least one hour before food intake. In some aspects, the method may comprise administering the composition before food intake, wherein the composition is administered at least 2 hours before food intake. In some aspects, the method may comprise administering the composition before food intake, wherein the composition is administered at least 3 hours before food intake. In some aspects, the method may comprise administering the composition before food intake, wherein the composition is administered at least 4 hours before food intake.

Embodiments include the methods of the preceding embodiments, wherein the microbes are administered with food intake.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

The content of the International Nucleotide Sequence Database Collaboration (DDBJ/EMBL/GENBANK) accession number CP001071.1 for microbial strain Akkermansia muciniphila, culture collection ATCC BAA-835, is herein incorporated by reference in its entirety.

The content of DDBJ/EMBL/GENBANK accession number AJ518871.2 for microbial strain Anaerofustis stercorihominis, culture collection DSM 17244, is herein incorporated by reference in its entirety.

The content of DDBJ/EMBL/GENBANK accession number DS499744.1 for microbial strain Anaerostipes caccae, culture collection DSM 14662, is herein incorporated by reference in its entirety.

The content of DDBJ/EMBL/GENBANK accession number AJ270487.2 for microbial strain Anaerostipes caccae, butyrate-producing bacterium L1-92, is herein incorporated by reference in its entirety.

The content of DDBJ/EMBL/GENBANK accession number AY305319.1 for microbial strain Anaerostipes hadrus, butyrate-producing bacterium SS2/1, is herein incorporated by reference in its entirety.

The content of DDBJ/EMBL/GENBANK accession number AJ315980.1 for microbial strain Anaerotruncus colihominis, culture collection DSM 17241, is herein incorporated by reference in its entirety.

The content of DDBJ/EMBL/GENBANK accession number GCA 000156195.1 for microbial strain, Bacteroides finegoldii, culture collection DSM 17565, is herein incorporated by reference in its entirety.

The content of DDBJ/EMBL/GENBANK accession number GCF 000154125.1 for microbial strain, Bacteroides ovatus, culture collection ATCC 8483, is herein incorporated by reference in its entirety.

The content of DDBJ/EMBL/GENBANK accession number NZ ABFZ00000000.2 for microbial strain, Bacteroides stercoris, culture collection ATCC 43183, is herein incorporated by reference in its entirety.

The content of DDBJ/EMBL/GENBANK accession number AP009256.1 for microbial strain, Bifidobacterium adolescentis, culture collection ATCC 15703, is herein incorporated by reference in its entirety.

The content of DDBJ/EMBL/GENBANK accession number CP001095.1 for microbial strain Bifidobacterium longum subsp. infantis, culture collection ATCC 15697, is herein incorporated by reference in its entirety.

The content of DDBJ/EMBL/GENBANK accession number CP001095.1 for microbial strain Bifidobacterium longum, culture collection ATCC 15697, is herein incorporated by reference in its entirety.

The content of DDBJ/EMBL/GENBANK accession number NZ_ACBZ00000000.1; for microbial strain Blautia hydrogenotrophica, culture collection DSM 10507, is herein incorporated by reference in its entirety.

The content of DDBJ/EMBL/GENBANK accession number ARET01 for microbial strain Blautia producta, culture collection ATCC 27340, is herein incorporated by reference in its entirety.

The content of DDBFEMBL/GenBank accession number U41172.1 for microbial strain Butyrivibrio fibrisolvens, culture collection ATCC 19171, is herein incorporated by reference in its entirety.

The content of DDBFEMBL/GenBank accession number AJ250365.2 for microbial strain Butyrivibrio fibrisolvens, 16.4, is herein incorporated by reference in its entirety.

The content of DDBFEMBL/GenBank accession number U41168.1 for microbial strain Butyrivibrio fibrisolvens, OB 156, is herein incorporated by reference in its entirety.

The content of DDBFEMBL/GenBank accession number AY305305.1 for microbial strain Butyrate-producing bacterium, A2-232, is herein incorporated by reference in its entirety.

The content of DDBFEMBL/GenBank accession number AY305316.1 for microbial strain Butyrate-producing bacterium, SS3/4, is herein incorporated by reference in its entirety.

The content of DDBJ/EMBL/GENBANK accession number AE001437.1 for microbial strain Clostridium acetobutylicum, culture collection ATCC 824, is herein incorporated by reference in its entirety.

The content of DDBJ/EMBL/GENBANK accession number X78070.1 for microbial strain Clostridium acetobutylicum, culture collection DSM 792, is herein incorporated by reference in its entirety.

The content of DDBJ/EMBL/GENBANK accession number CP000721.1 for microbial strain Clostridium beijerinckii, culture collection NCIMB 8052, is herein incorporated by reference in its entirety.

The content of DDBJ/EMBL/GENBANK accession number X68189.1 for microbial strain Clostridium sporogenes, culture collection DSM 795, is herein incorporated by reference in its entirety.

The content of DDBJ/EMBL/GENBANK accession number X74770.1 for microbial strain Clostridium tetani, is herein incorporated by reference in its entirety.

The content of DDBJ/EMBL/GENBANK accession number AE001437.1 for microbial strain Collinsella aerofaciens, culture collection ATCC 25986, is herein incorporated by reference in its entirety.

The content of DDBJ/EMBL/GENBANK accession number AJ270491.2 for microbial strain Coprococcus, butyrate-producing bacterium L2-50, is herein incorporated by reference in its entirety.

The content of DDBJ/EMBL/GENBANK accession number ABVR01 for microbial strain Coprococcus comes, culture collection ATCC 27758, is herein incorporated by reference in its entirety.

The content of DDBJ/EMBL/GENBANK accession number EF031543.1 for microbial strain Coprococcus eutactus, culture collection ATCC 27759, is herein incorporated by reference in its entirety.

The content of DDBFEMBL/GenBank accession number AY305306.1 for microbial strain Eubacterium cylindroides, butyrate-producing bacterium T2-87, is herein incorporated by reference in its entirety.

The content of DDBFEMBL/GenBank accession number AY305313.1 for microbial strain Eubacterium cylindroides, butyrate-producing bacterium SM7/11, is herein incorporated by reference in its entirety.

The content of DDBFEMBL/GenBank accession number L34682.2 for microbial strain Eubacterium dolichum, culture collection DSM 3991, is herein incorporated by reference in its entirety.

The content of DDBFEMBL/GenBank accession number AJ270490.2 for microbial strain Eubacterium hallii, butyrate-producing bacterium L2-7, is herein incorporated by reference in its entirety.

The content of DDBFEMBL/GenBank accession number AY305318.1 for microbial strain Eubacterium hallii, butyrate-producing bacterium SM6/1, is herein incorporated by reference in its entirety.

The content of DDBFEMBL/GenBank accession number L34621.2 for microbial strain Eubacterium hallii, culture collection ATCC 27751, is herein incorporated by reference in its entirety.

The content of DDBJ/EMBL/GenBank accession number NZ_CP019962.1 for microbial strain Eubacterium limosum, culture collection ATCC 5486, is herein incorporated by reference in its entirety.

The content of DDBFEMBL/GenBank accession number AJ270475.2 for microbial strain Eubacterium rectale, A1-86, is herein incorporated by reference in its entirety.

The content of DDBJ/EMBL/GENBANK accession number NC 012781.1 for microbial strain Eubacterium rectale, culture collection ATCC 33656, is herein incorporated by reference in its entirety.

The content of DDBFEMBL/GenBank accession number L34421.2 for microbial strain Eubacterium ventriosum, culture collection ATCC 27560, is herein incorporated by reference in its entirety.

The content of DDBJ/EMBL/GENBANK accession number AY305307.1 for microbial strain Faecalibacterium prausnitzii, butyrate producing bacterium M21/2, is herein incorporated by reference in its entirety.

The content of DDBJ/EMBL/GENBANK accession number FP929046.1 for microbial strain Faecalibacterium prausnitzii is herein incorporated by reference in its entirety.

The content of DDBJ/EMBL/GENBANK accession number GG697168.2 for microbial strain Faecalibacterium prausnitzii is herein incorporated by reference in its entirety.

The content of DDBJ/EMBL/GENBANK accession number CP002158.1 for microbial strain Fibrobacter succino genes subsp. succino genes is herein incorporated by reference in its entirety.

The content of DDBJ/EMBL/GENBANK accession number NZ_AUJN01000001.1 for microbial strain Clostridium butyricum, culture collection DSM 10702 is herein incorporated by reference in its entirety.

The content of DDBJ/EMBL/GENBANK accession number NZ AZUI01000001.1 for microbial strain Clostridium indolis, culture collection DSM 755, is herein incorporated by reference in its entirety.

The content of DDBJ/EMBL/GENBANK accession number ACEP01000175.1 for microbial strain Eubacterium hallii, culture collection DSM 3353, is herein incorporated by reference in its entirety.

The content of DDBFEMBL/GenBank accession number AY305310.1 for microbial strain Roseburia faecis, M72/1, is herein incorporated by reference in its entirety.

The content of DDBFEMBL/GenBank accession number AJ270482.2 for microbial strain Roseburia hominis, type strain A2-183T, is herein incorporated by reference in its entirety.

The content of DDBFEMBL/GenBank accession number AJ312385.1 for microbial strain Roseburia intestinalis, L1-82, is herein incorporated by reference in its entirety.

The content of DDBFEMBL/GenBank accession number AJ270473.3 for microbial strain Roseburia inulinivorans, type strain A2-194T, is herein incorporated by reference in its entirety.

The content of DDBJ/EMBL/GENBANK accession number NZ_ACFY01000179.1 for microbial strain Roseburia inulinivorans, culture collection DSM 16841, is herein incorporated by reference in its entirety.

The content of DDBJ/EMBL/GENBANK accession number K1912489.1 for microbial strain Ruminococcus flavefaciens, culture collection ATCC 19208, is herein incorporated by reference in its entirety.

The content of DDBJ/EMBL/GENBANK accession number AAYG02000043.1 for microbial strain Ruminococcus gnavus, culture collection ATCC 29149, is herein incorporated by reference in its entirety.

### BRIEF DESCRIPTION OF THE DRAWINGS

The patent application contains at least one drawing executed in color. Copies of this patent or patent application with color drawings will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1a depicts reduced visceral hypersensitivity in treated and control male mice in a neonatal model.

FIG. 1B depicts visceral motor reflex (VMR) response to CRD after 1 week of withdrawal for treated and control male mice in an IBS model.

FIG. 2a depicts VMR response for treated and control mice in an IBS model.

FIG. 2b depicts VMR response for treated and control older female mice after 1 week of withdrawal in an IBS model.

FIG. 3 depicts VMR response for treated and control mice in an IBS model after treatment had been withdrawn for one week.

FIG. 4 depicts EMG responses to colorectal distension in treated and untreated mice in a model of IBS.

FIG. 5 depicts a representation of an open plus table used for testing and the amount of time spent on the open arms for treated and untreated mice in a model of IBS.

FIG. 6 depicts the experimental design for measuring TRPV1 activity in treated and untreated mice in a model of IBS.

FIG. 7 depicts TRPV1 activity in treated, untreated, and withdrawn from treatment mice in a model of IBS.

FIG. 8 depicts nNOS and HuC/D staining in LMMP in treated and untreated mice in a model of IBS.

FIG. 9 depicts the quantification of nNOS and HuC/D staining in LMMP in treated and untreated mice in a model of IBS.

### DETAILED DESCRIPTION

### Definitions

As used in the specification and claims, the singular forms “a”, “an” and “the” include plural references unless the context clearly dictates otherwise. For example, the term “a sample” includes a plurality of samples, including mixtures thereof.

The terms “microbes” and “microorganisms” are used interchangeably herein and can refer to bacteria, archaea, eukaryotes (e.g. protozoa, fungi, yeast), and viruses, including bacterial viruses (i.e. phage).

The term “microbiome”, “microbiota”, and “microbial habitat” are used interchangeably herein and can refer to the ecological community of microorganisms that live on or in a subject's body. The microbiome can be comprised of commensal, symbiotic, and/or pathogenic microorganisms. Microbiomes can exist on or in many, if not most parts of the subject. Some non-limiting examples of habitats of microbiome can include: body surfaces, body cavities, body fluids, the gut, the colon, skin surfaces and pores, vaginal cavity, umbilical regions, conjunctival regions, intestinal regions, the stomach, the nasal cavities and passages, the gastrointestinal tract, the urogenital tracts, saliva, mucus, and feces.

The term “pharmaceutical formulation” is any composition or formulation designed for administration to a subject. Such formulations may or may not meet the safety, efficacy, or other requirements for human use or approval by the FDA or other approval body or institution.

The term “prebiotic” as used herein can be a general term to refer to chemicals and or ingredients that can affect the growth and/or activity of microorganisms in a host (e.g. can allow for specific changes in the composition and/or activity in the microbiome). Prebiotics can confer a health benefit on the host. Prebiotics can be selectively fermented, e.g. in the colon. Some non-limiting examples of prebiotics can include: complex carbohydrates, complex sugars, resistant dextrins, resistant starch, amino acids, peptides, nutritional compounds, biotin, polydextrose, fructooligosaccharide (FOS), galactooligosaccharides (GOS), inulin, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), green banana, reishi, tapioca, oats, pectin, potato or extracts thereof. Prebiotics can be found in foods (e.g. acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. Prebiotics can also be administered in other forms (e.g. capsule or dietary supplement).

The term “probiotic” as used herein can mean one or more microorganisms which, when administered appropriately, can confer a health benefit on the host or subject. Some non-limiting examples of probiotics include: Akkermansia mucimphila, Anaerostipes caccae, Bacteroides finegoldii, Bacteroides ovatus, Bacteroides stercoris, Eubactrium hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium butyricum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof.

The terms “determining”, “measuring”, “evaluating”, “assessing,” “assaying,” and “analyzing” can be used interchangeably herein and can to refer to any form of measurement, and include determining if an element is present or not. (e.g., detection). These terms can include both quantitative and/or qualitative determinations. Assessing may be relative or absolute. These terms can include use of the algorithms and databases described herein. “Detecting the presence of” can include determining the amount of something present, as well as determining whether it is present or absent. The term “genome assembly algorithm” as used herein, refers to any method capable of aligning sequencing reads with each other (de novo) or to a reference (re-sequencing) under conditions that a complete sequence of the genome may be determined.

The term “genome” as used herein, can refer to the entirety of an organism's hereditary information that is encoded in its primary DNA sequence. The genome includes both the genes and the non-coding sequences. For example, the genome may represent a microbial genome. The genetic content of the microbiome can comprise: genomic DNA, RNA, and ribosomal RNA, the epigenome, plasmids, and all other types of genetic information found in the microbes that comprise the microbiome.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand. The nucleic acid sequence can be made up of adenine, guanine, cytosine, thymine, and uracil (A, T, C, G, and U) as well as modified versions (e.g. N6-methyladenosine, 5-methylcytosine, etc.).

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence.

The term “sequencing” as used herein refers to sequencing methods for determining the order of the nucleotide bases—A, T, C, G, and U—in a nucleic acid molecule (e.g., a DNA or RNA nucleic acid molecule.

The term “biochip” or “array” can refer to a solid substrate having a generally planar surface to which an adsorbent is attached. A surface of the biochip can comprise a plurality of addressable locations, each of which location may have the adsorbent bound there. Biochips can be adapted to engage a probe interface, and therefore, function as probes. Protein biochips are adapted for the capture of polypeptides and can be comprise surfaces having chromatographic or biospecific adsorbents attached thereto at addressable locations. Microarray chips are generally used for DNA and RNA gene expression detection.

The term “barcode” as used herein, refers to any unique, non-naturally occurring, nucleic acid sequence that may be used to identify the originating genome of a nucleic acid fragment.

The terms “subject,” “individual,” “host,” and “patient” can be used interchangeably herein and refer to any animal subject, including: humans, mammals, laboratory animals, livestock, and household pets. The subject can host a variety of microorganisms. The subject can have different microbiomes in various habitats on and in their body. The subject may be diagnosed or suspected of being at high risk for a disease. The subject may have a microbiome state that is contributing to a disease (a dysbiosis). In some cases, the subject is not necessarily diagnosed or suspected of being at high risk for the disease. In some instances a subject may be suffering from an infection or at risk of developing or transmitting to others an infection.

The terms “treatment” or “treating” are used interchangeably herein. These terms can, but not necessarily, refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The terms “16S”, “16S ribosomal subunit”, and “16S ribosomal RNA (rRNA)” can be used interchangeably herein and can refer to a component of a small subunit (e.g., 30S) of a prokaryotic (e.g., bacteria, archaea) ribosome. The 16S rRNA is highly conserved evolutionarily among species of microorganisms. Consequently, sequencing of the 16S ribosomal subunit can be used to identify and/or compare microorganisms present in a sample (e.g., a microbiome).

The term “spore” as used herein can refer to a viable cell produced by a microorganism to resist unfavorable conditions such as high temperatures, humidity, and chemical agents. A spore can have thick walls that allow the microorganism to survive harsh conditions for extended periods of time. Under suitable environmental conditions, a spore can germinate to produce a living form of the microorganism that is capable of reproduction and all of the physiological activities of the microorganism.

The term “protein component” refers to one or more proteins or fragments thereof that are extracted, isolated, derived, and/or purified from one or more microbes of the disclosure. A protein component can comprise an isolated protein, fragment, or derivative thereof, or a mixture of any two or more proteins, fragments, or derivatives thereof. In some embodiments, a protein component can retain functional properties or beneficial properties even after isolation from a microbe, for example, functional properties or beneficial properties exhibited by a microbe of the disclosure or a protein expressed by a microbe of the disclosure. A protein component can be or can comprise a protein or fragment thereof from a secreted protein, a membrane protein, an inner membrane protein, an outer membrane protein, a periplasmic protein, a cell wall protein, or a cytoplasmic protein. In some embodiments, the protein component comprises an amino acid sequence from a wild type protein. In some embodiments, the protein component comprises an amino acid sequence from a variant of a wild type protein, for example, a sequence with one or more amino acid insertions, deletions, and/or substitutions relative to an amino acid sequence from the wild type protein. In some embodiments, the protein component comprises an amino acid sequence with at least 60%, at least 70%, at least 80%, at least 85%, at least 90%, at least 95%, at least 96%, at least 97%, at least 98%, at least 99%, at least 99.5%, or at least 99.9% sequence identity to an amino acid sequence from a wild type protein. The protein component can comprise a post translational modification, for example, acetylation, amidation, biotinylation, deamidation, farnesylation, formylation, fucosylation, geranylgeranylation, glutathionylation, glycation, glycosylation, hydroxylation, methylation, mono-ADP-ribosylation, myristoylation, N-acetylation, N-glycosylation, N-myristoylation, nitrosylation, oxidation, palmitoylation, phosphorylation, poly(ADP-ribosyl)ation, sialylation, stearoylation, sulfation, SUMOylation, ubiquitiniation, or any combination thereof. A protein component can comprise, for example, a lipoprotein, a glycoprotein, or a phosphoprotein. A protein component can be obtained, for example, by harvesting the supernatant of a microbial culture, and/or extracting the protein component from a culture of a microbe as disclosed herein. In some embodiments, a protein component is produced using an expression system, for example, expression of a recombinant protein by a suitable host cell, or a cell-free biosynthetic process. A protein component can be extracted, isolated, or derived from a microbe of the disclosure using any suitable technique, including without limitation chromatographic methods, size exclusion chromatography, hydrophobic interaction chromatography, ion exchange chromatography, affinity chromatography, immunoaffinity chromatography, metal binding, immunoprecipitation, HPLC, ultracentrifugation, precipitation and differential solubilization, and extraction. In some embodiments, a protein component is obtained after pasteurization of a microbe as disclosed herein. In some embodiments, enzymes are used in making a protein component of the disclosure, for example, a protease, or an enzyme that introduces a post-translational modification. A protein component can be a degradation product, for example, a protein fragment generated by cleavage of a larger protein. Disclosure herein related to microbes can also apply to protein fragments from the microbes. For example, in some embodiments the disclosure provides compositions comprising protein component(s) from any one or more microorganisms disclosed herein, e.g., pharmaceutical formulations, therapeutic compositions, dietary supplements, nutritional supplements, medical probiotics, or medical foods. Disclosure herein related to compositions and formulations comprising microorganisms can also apply to protein components from those microoganisms. For example formulations, dosage forms, routes of administration, coatings (e.g., enteric coatings), encapsulation, methods of treatment, etc. as disclosed herein can comprise protein component(s) from one or more microbes of the disclosure.

The term “homoacetogen” or acetogen refers to a microorganism that generates acetate (CH3COO—) as an end product of anaerobic respiration or fermentation. In some embodiments, the microorganisms are bacteria. These microbes perform anaerobic respiration and carbon fixation simultaneously through the reductive acetyl coenzyme A (acetyl-CoA) pathway (also known as the Wood-Ljungdahl pathway).

Compositions comprising microbes such as probiotics can confer a variety of beneficial effects on a subject. Examples of these beneficial effects can include reduction of pain, immunomodulatory features, regulation of cell proliferation, the ability to promote normal physiologic development of the mucosal epithelium, and enhancement of human nutrition. Microbial-based compositions can be administered as a therapeutic to a subject suffering from a microbiome-related health condition or disorder. Microbial-based compositions can be administered as a therapeutic to a subject so as to treat one or more disorders which are not related to the microbiome. In some embodiments, microbial compositions and microbial-based compositions include compositions that comprise a protein component of one or more microbes disclosed herein.

Microbial Compositions and Formulations

Compositions or formulations of the disclosure can be administered as pharmaceutical formulations, therapeutic composition, dietary supplements, nutritional supplements, medical probiotics, or a medical food. In some cases, the composition is administered as a pharmaceutical formulation. In some cases, the composition is administered as a nutritional supplement. In some cases, the composition is administered as a dietary supplement. In some cases, the composition is administered as a medical food. In some cases, the composition is administered as a medical probiotic. In some cases, a composition (e.g., a dietary supplement, a nutritional supplement, a medical probiotic, or a medical food) can be administered orally, for example, as a capsule, pill, or tablet.

In embodiments, disclosed herein are formulations comprising one or more microbes selected from the group consisting of Akkermansia sp., Anaerostipes sp., Bacteroides sp., Bifidobacterium sp., Blautia sp., Clostridium sp., Collinsella sp., Coprococcus sp., Eubacterium sp., and Ruminococcus sp., and any combination thereof. In embodiments, a formulation comprises a protein component from one or more microbes selected from the group consisting of Akkermansia sp., Anaerostipes sp., Bacteroides sp., Bifidobacterium sp., Blautia sp., Clostridium sp., Collinsella sp., Coprococcus sp., Eubacterium sp., and Ruminococcus sp., and any combination thereof. As used herein, “sp.” stands for “species” and refers to all species of the recited genus that the term follows. Included are compositions comprising 1, 2, 3, 4, 5, 6, 7, 8, 9, or all 10 of the different genera recited, or protein component(s) therefrom. In further embodiments, such formulations can be used to treat or manage a gut disorder. Examples of such gut disorders include, but are not limited to, irritable bowel syndrome, inflammatory bowel disease, ulcerative colitis, diarrhea, constipation, leaky intestine, and/or Crohn's disease. In embodiments, the formulations reduce visceral motor reflex in the colon and/or reduce pain in the colon of a subject treated with the formulation.

In embodiments, disclosed herein are formulations comprising one or more microbes selected from the group consisting of Akkermansia muciniphila, Anaerostipes caccae, Bacteroides finegoldii, Bacteroides ovatus, Bacteroides stercoris, Bifidobacterium adolescentis, Bifidobacterium infantis, Bifidobacterium faecale, Bifidobacterium longum, Blautia hydrogenotrophica, Blautia producta, Clostridium butyricum, Clostridium beijerinckii, Clostridium innocuum, Clostridium sporogenes, Collinsella aerofaciens, Coprococcus comes, Eubacterium hallii, Eubacterium limosum, and Ruminococcus faecis and any combination thereof. In embodiments, disclosed herein are formulations comprising a protein component from one or more microbes selected from the group consisting of Akkermansia muciniphila, Anaerostipes caccae, Bacteroides finegoldii, Bacteroides ovatus, Bacteroides stercoris, Bifidobacterium adolescentis, Bifidobacterium infantis, Bifidobacterium faecale, Bifidobacterium longum, Blautia hydrogenotrophica, Blautia producta, Clostridium butyricum, Clostridium beijerinckii, Clostridium innocuum, Clostridium sporogenes, Collinsella aerofaciens, Coprococcus comes, Eubacterium hallii, Eubacterium limosum, and Ruminococcus faecis and any combination thereof. Included are compositions comprising 1, 2, 3, 4, 5, 6, 7, 8, 9, 10, 11, 12, 13, 14, 15, 16, 17, 18, 19 or all 20 of the different species recited and any combination thereof, or protein component(s) therefrom. In further embodiments, such formulations can be used to treat a gut disorder. Examples of such gut disorders include, but are not limited to, irritable bowel syndrome, inflammatory bowel disease, ulcerative colitis, diarrhea, constipation, leaky intestine, and/or Crohn's disease. In embodiments, the formulations reduce visceral motor reflex in the colon and/or reduce pain in the colon of a subject treated with the formulation.

In embodiments, disclosed herein are formulations comprising one or more microbes having a 16S rRNA sequence comprising at least 95% identity to the full length of a 16S rRNA sequence of a microbe selected from the group consisting of Akkermansia muciniphila ATCC BAA-835, Anaerostipes caccae DSM 14662, Bacteroides finegoldii DSM 17565, Bacteroides ovatus ATCC 8483, Bacteroides stercoris ATCC 43183, Bifidobacterium adolescentis ATCC 15703, Bifidobacterium infantis ATCC 15697, Bifidobacterium faecale JCM 19861, Bifidobacterium longum ATCC 15697, Blautia hydrogenotrophica DSM 10507, Blautia producta ATCC 27340, Clostridium butyricum DSM 10702, Clostridium beijerinckii NCIMB 8052, Clostridium innocuum ATCC 14501, Clostridium sporogenes DSM 795, Collinsella aerofaciens ATCC 25986, Coprococcus comes ATCC 27758, Eubacterium hallii DSM 3353, Eubacterium limosum ATCC 5486, and Ruminococcus faecis JCM 15917 and any combination thereof, or protein components therefrom. Included are compositions comprising 1, 2, 3, 4, 5, 6, 7, 8, 9, 10, 11, 12, 13, 14, 15, 16, 17, 18, 19 or all 20 different microbes such that 1, 2, 3, 4, 5, 6, 7, 8, 9, 10, 11, 12, 13, 14, 15, 16, 17, 18, 19 or all 20 of the different 16S rRNA sequences are present in the composition. Certain embodiments include formulations, wherein the at least about 95% sequence identity is selected from the group consisting of: at least about 96%, at least about 97%, at least about 98%, at least about 99%, at least about 99.5%, and at least about 99.5% sequence identity to a rRNA sequence. In further embodiments, such formulations can be used to treat a gut disorder. Examples of such gut disorders include, but are not limited to, irritable bowel syndrome, inflammatory bowel disease, ulcerative colitis, diarrhea, constipation, leaky intestine, and/or Crohn's disease. In embodiments, the formulations reduce visceral motor reflex in the colon and/or reduce pain in the colon of a subject treated with the formulation.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Anaerostipes caccae DSM

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Bifidobacterium adolescentis ATCC 15703.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Ruminococcus faecis JCM 15917.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Blautia producta ATCC 27340.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Coprococcus comes ATCC 27758.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Akkermansia muciniphila ATCC BAA-835.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Bacteroides finegoldii DSM 17565.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Bacteroides ovatus ATCC 8483.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Bacteroides stercoris ATCC 43183.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Bifidobacterium infantis ATCC 15697.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Bifidobacterium faecale JCM 19861.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Bifidobacterium longum ATCC 15697.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Blautia hydrogenotrophica DSM 10507.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Clostridium butyricum DSM 10702.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Clostridium beijerinckii NCIMB 8052.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Clostridium innocuum ATCC 14501.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Clostridium sporogenes DSM 795.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Collinsella aerofaciens ATCC 25986.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Eubacterium hallii DSM 3353.

In one embodiment, a composition to treat a gut disorder comprises an isolated and/or purified microorganism population consisting of microbes with at least about: 95%, 96%, 97%, 98%, 99%, 99.5%, or 100% sequence identity to the 16SrRNA of Eubacterium limosum ATCC 5486.

In one embodiment, a composition to treat a metabolic disorder comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Akkermansia muciniphila.

In one embodiment, a composition to treat a metabolic disorder comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Anaerostipes caccae.

In one embodiment, a composition to treat a metabolic disorder comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Bacteroides finegoldii.

In one embodiment, a composition to treat a metabolic disorder comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Bacteroides ovatus.

In one embodiment, a composition to treat a metabolic disorder comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Bacteroides stercoris.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Bifidobacterium adolescentis.

In one embodiment, a composition comprises an isolated and/or purified the 16SrRNA of Bifidobacterium bifidum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Bifidobacterium infantis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Bifidobacterium faecale.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Bifidobacterium longum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Blautia hydrogenotrophica.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Blautia producta.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Butyrivibrio fibrisolvens.

In one embodiment, a composition comprises an isolated and/or purified microorganism population consisting of microbes with at least about: 70%, 75%, 80%, 85%, 87%, the 16SrRNA of Clostridium acetobutylicum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium aminophilum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium beijerinckii.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium butyricum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium colinum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium indolis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium innocuum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium orbiscindens.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Collinsella aerofaciens.

In one embodiment, a composition comprises an isolated and/or purified the 16SrRNA of Coprococcus comes.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Enterococcus faecium.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Eubacterium hallii.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Eubacterium limosum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Eubacterium rectale.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Faecalibacterium prausnitzii.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Fibrobacter succinogenes.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Lactobacillus acidophilus.

In one embodiment, a composition comprises an isolated and/or purified microorganism population consisting of microbes with at least about: 70%, 75%, 80%, 85%, 87%, the 16SrRNA of Lactobacillus brevis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Lactobacillus bulgaricus.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Lactobacillus casei.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Lactobacillus caucasicus.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Lactobacillus fermentum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Lactobacillus helveticus.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Lactobacillus lactis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Lactobacillus plantarum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Lactobacillus reuteri.

In one embodiment, a composition comprises an isolated and/or purified the 16SrRNA of Lactobacillus rhamnosus.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Oscillospira guilliermondii.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Roseburia cecicola.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Roseburia inulinivorans.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Ruminococcus faecis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Ruminococcus flavefaciens.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Ruminococcus gnavus.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Ruminococcus obeum.

In one embodiment, a composition comprises an isolated and/or purified microorganism population consisting of microbes with at least about: 70%, 75%, 80%, 85%, 87%, the 16SrRNA of Streptococcus cremoris.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA Streptococcus faecium.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Streptococcus infantis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Streptococcus mutans.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Streptococcus thermophilus.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Anaerofustis stercorihominis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Anaerostipes hadrus.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Anaerotruncus colihominis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium sporogenes.

In one embodiment, a composition comprises an isolated and/or purified the 16SrRNA of Clostridium sporogenes.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium tetani.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Coprococcus.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Coprococcus eutactus.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Eubacterium cylindroides.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Eubacterium dolichum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Eubacterium ventriosum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Roseburia faeccis.

In one embodiment, a composition comprises an isolated and/or purified microorganism population consisting of microbes with at least about: 70%, 75%, 80%, 85%, 87%, the 16SrRNA of Roseburia hominis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Roseburia intestinalis.

In one embodiment, a composition comprises microbes from 2 or more, 3 or more, four or more, or all five of the group consisting of Anaerostipes caccae, Bifidobacterium adolescentis, Blautia producta, Coprococcus comes, and Ruminococcus faecis.

In one embodiment, a composition comprises 2 or more, 3 or more, four or more, or all five different microbes of the group consisting of microbes having 16S rRNA sequence comprising at least 97% identity to the full length of a 16S rRNA sequence of Anaerostipes caccae, Bifidobacterium adolescentis, Blautia producta, Coprococcus comes, and Ruminococcus faecis.

In one embodiment, a composition comprises microbes from 2 or more, 3 or more, four or more, or all five of the group consisting of Anaerostipes caccae DSM 14662, Bifidobacterium adolescentis ATCC 15703, Blautia producta ATCC 27340, Coprococcus comes ATCC 27758, and Ruminococcus faecis JCM 15917.

In one embodiment, a composition comprises Anaerostipes caccae, Bifidobacterium adolescentis, Blautia producta, Coprococcus comes, and Ruminococcus faecis.

In one embodiment, a composition comprises 16S rRNA sequence comprising at least 97% identity to the full length of a 16S rRNA sequence of Anaerostipes caccae, Bifidobacterium adolescentis, Blautia producta, Coprococcus comes, and Ruminococcus faecis.

In one embodiment, a composition comprises Anaerostipes caccae DSM 14662, Bifidobacterium adolescentis ATCC 15703, Blautia producta ATCC 27340, Coprococcus comes ATCC 27758, and Ruminococcus faecis JCM 15917.

In one embodiment, a composition comprises Anaerostipes caccae, and Coprococcus comes.

In one embodiment, a composition comprises 16S rRNA sequence comprising at least 97% identity to the full length of a 16S rRNA sequence of Anaerostipes caccae, and Coprococcus comes.

In one embodiment, a composition comprises Anaerostipes caccae DSM 14662, and Coprococcus comes ATCC 27758.

In one embodiment, a composition comprises Anaerostipes caccae, Blautia producta, and Coprococcus comes.

In one embodiment, a composition comprises 16S rRNA sequence comprising at least 97% identity to the full length of a 16S rRNA sequence of Anaerostipes caccae, Blautia producta, and Coprococcus comes.

In one embodiment, a composition comprises Anaerostipes caccae DSM 14662, Blautia producta ATCC 27340, and Coprococcus comes ATCC 27758.

Certain embodiments include a composition of any of the preceding embodiments, wherein the formulation further comprises one or more additional microbe strains having a 16S rRNA sequence comprising at least 95% identity to the full length of a 16S rRNA sequence of a microbe selected from the group consisting of Akkermansia muciniphila, Anaerostipes caccae, Bacteroides finegoldii, Bacteroides ovatus, Bacteroides stercoris, Eubactrium hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium butyricum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof. Certain embodiments include formulations, wherein the at least about 95% sequence identity is selected from the group consisting of: at least about 96%, at least about 97%, at least about 98%, at least about 99%, at least about 99.5%, and at least about 99.5% sequence identity to a rRNA sequence.

A composition may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, or at least 50, or at least 75, or at least 100 types of microbes. A composition may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 45, or at most 50, or at most 75, or at most 100 types of microbes.

Provided herein are compositions that may be administered as pharmaceuticals, therapeutics, dietary or nutritional supplements, and/or cosmetics. One or more microorganisms or protein components therefrom described herein can be used to create a composition comprising an effective amount of the composition for treating a subject. The microorganisms or protein components therefrom can be in any formulation known in the art. Some non-limiting examples can include topical, capsule, pill, enema, liquid, injection, and the like. In some embodiments, the one or more strains disclosed herein may be included in a food or beverage product, cosmetic, or nutritional supplement.

In some embodiments, a composition as described herein comprises an enteric coating. The composition may be formulated as an enteric-coated pill. An enteric-coating can protect the contents of a formulation, for example, pill or capsule, from the acidity of the stomach and provide delivery to the ileum and/or upper colon regions. Non-limiting examples of enteric coatings include pH sensitive polymers (e.g., eudragit FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers.

The enteric coating can be designed to dissolve at any suitable pH. In some embodiments, the enteric coating is designed to dissolve at a pH greater than about pH 6.5 to about pH 7.0. In some embodiments, the enteric coating is designed to dissolve at a pH greater than about pH 6.5. In some embodiments, the enteric coating is designed to dissolve at a pH greater than about pH 7.0. The enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units.

A composition can be substantially free of preservatives. In some applications, the composition may contain at least one preservative. In particular embodiments, formulations as described herein may contain an effective amount of a preservative. An “effective” amount is any amount that preserves or increases the shelf life of the composition beyond what would be obtained if the preservative were not present in the formulation. Examples of such preservatives include, but are not limited to, Vitamin E. Vitamin C, butylmedhydroxyanisole (BHA), butylatedhydroxytoluene (BHT), disodium ethylenediaminetetraacetic acid (EDTA), polyphosphates, citric acid, benzoates, sodium benzoate, sorbates, propionets, and nitrites.

The formulation can include one or more active ingredients. Active ingredients include, but are not limited to, antibiotics, prebiotics, probiotics, glycans (e.g., as decoys that would limit specific bacterial/viral binding to the intestinal wall), bacteriophages, microorganisms, bacteria, protein components, and the like.

In some embodiments, the formulation comprises a prebiotic. In some embodiments, the prebiotic is inulin, green banana, reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof. The prebiotic can serve as an energy source for the microbial formulation.

A formulation can be formulated for administration by a suitable method for delivery to any part of the gastrointestinal tract of a subject including oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract.

Pharmaceutical formulations can be formulated as a dietary supplement. Pharmaceutical formulations can be incorporated with vitamin supplements. pharmaceutical formulations can be formulated in a chewable form such as a probiotic gummy. Pharmaceutical formulations can be incorporated into a form of food and/or drink. Non-limiting examples of food and drinks where the microbial compositions can be incorporated include, for example, bars, shakes, juices, infant formula, beverages, frozen food products, fermented food products, and cultured dairy products such as yogurt, yogurt drink, cheese, acidophilus drinks, and kefir.

A formulation of the disclosure can be administered as part of a fecal transplant process. A formulation can be administered to a subject by a tube, for example, nasogastric tube, nasojejunal tube, nasoduodenal tube, oral gastric tube, oral jejunal tube, or oral duodenal tube. A formulation can be administered to a subject by colonoscopy, endoscopy, sigmoidoscopy, and/or enema.

In some embodiments, the composition is formulated such that the one or more microbes can replicate once they are delivered to the target habitat (e.g. the gut). In one non-limiting example, the microbial composition is formulated in a capsule or a pill, such that the capsule or pill has a shelf life of at least 1, 2, 3, 4, 5, 6, 7, 8, 9, 10, 11, or 12 months. In another non-limiting example, the storage of the microbial composition is formulated so that the microbes can reproduce once they are in the gut. In some embodiments, other components may be added to aid in the shelf life of the microbial composition. In some embodiments, one or more microbes may be formulated in a manner that it is able to survive in a non-natural environment. For example, a microbe that is native to the gut may not survive in an oxygen-rich environment. To overcome this limitation, the microbe may be formulated in a pill that can reduce or eliminate the exposure to oxygen. Other strategies to enhance the shelf-life of microbes may include other microbes (e.g. if the composition comprises elements whereby one or more strains is helpful for the survival of one or more strains).

In some embodiments, one or more of the microbes are lyophilized (e.g., freeze-dried) and formulated as a powder, tablet, enteric-coated capsule (e.g. for delivery to ileum/colon), or pill that can be administered to a subject by any suitable route. The lyophilized formulation can be mixed with a saline or other solution prior to administration.

In some embodiments, a composition is formulated for oral administration, for example, as an enteric-coated capsule or pill, for delivery of the contents of the formulation to the ileum and/or colon regions of a subject.

In some embodiments, the composition is formulated for oral administration. In some embodiments, the composition is formulated as an enteric-coated pill or capsule for oral administration. In some embodiments, the composition is formulated for delivery of the microbes or protein components therefrom to the ileum region of a subject. In some embodiments, the composition is formulated for delivery of the microbes or protein components therefrom to the colon region (e.g. upper colon) of a subject. In some embodiments, the composition is formulated for delivery of the microbes or protein components therefrom to the ileum and colon regions of a subject.

In some embodiments, the administration of a formulation of the disclosure can be preceded by, for example, colon cleansing methods such as colon irrigation/hydrotherapy, enema, administration of laxatives, dietary supplements, dietary fiber, enzymes, and magnesium.

In some embodiments, the composition is formulated as a population of spores. Spore-containing formulations can be administered by any suitable route described herein. Orally administered spore-containing formulations can survive the low pH environment of the stomach. The amount of spores employed can be, for example, from about 1% w/w to about 99% w/w of the entire formulation.

Formulations provided herein can include the addition of one or more agents to the therapeutics or cosmetics in order to enhance stability and/or survival of microbes in the formulation. Non-limiting example of stabilizing agents include genetic elements, glycerin, ascorbic acid, skim milk, lactose, tween, alginate, xanthan gum, carrageenan gum, mannitol, palm oil, and poly-L-lysine (POPL). In some embodiments, a stabilizing agent enhances the stability of a protein component.

In some embodiments, a formulation comprises one or more recombinant microbes or microbes that have been genetically modified. In other embodiments, one or more microbes are not modified or recombinant. In some embodiments, the formulation comprises microbes that can be regulated, for example, a microbe comprising an operon or promoter to control microbial growth. Microbes as described herein can be produced, grown, or modified using any suitable methods, including recombinant methods. A protein component can be from a genetically-modified microbe.

A formulation can be customized for a subject. A custom formulation can comprise, for example, a prebiotic, a probiotic, an antibiotic, or a combination of active agents described herein. Data specific to the subject comprising for example age, gender, and weight can be combined with an analysis result to provide a therapeutic agent customized to the subject. For example, a subject's microbiome found to be low in a specific microbe relative to a sub-population of healthy subjects matched for age and gender can be provided with a therapeutic and/or cosmetic formulation comprising the specific microbe to match that of the sub-population of healthy subjects having the same age and gender as the subject.

Formulations provided herein can include those suitable for oral including buccal and sub-lingual, intranasal, topical, transdermal, transdermal patch, pulmonary, vaginal, rectal, suppository, mucosal, systemic, or parenteral including intramuscular, intraarterial, intrathecal, intradermal, intraperitoneal, subcutaneous, and intravenous administration or in a form suitable for administration by aerosolization, inhalation or insufflation.

A formulation can include carriers and/or excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, antioxidants, gums, coating agents, coloring agents, flavoring agents, dispersion enhancer, disintegrant, coating agents, plasticizers, preservatives, suspending agents, emulsifying agents, plant cellulosic material and spheronization agents, and any combination thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A pharmaceutical, therapeutic, nutritional, dietary, or cosmetic composition can be encapsulated within a suitable vehicle, for example, a liposome, a microspheres, or a microparticle. Microspheres formed of polymers or proteins can be tailored for passage through the gastrointestinal tract directly into the blood stream. Alternatively, the compound can be incorporated and the microspheres, or composite of microspheres, and implanted for slow release over a period of time ranging from days to months.

A pharmaceutical, therapeutic, or cosmetic composition can be formulated as a sterile solution or suspension. The compositions can be sterilized by conventional techniques or may be sterile filtered. The resulting aqueous solutions may be packaged for use as is, or lyophilized. The lyophilized preparation of the microbial composition can be packaged in a suitable form for oral administration, for example, capsule or pill.

The compositions can be administered topically and can be formulated into a variety of topically administrable compositions, such as solutions, suspensions, lotions, gels, pastes, medicated sticks, balms, creams, and ointments. Such compositions can contain solubilizers, stabilizers, tonicity enhancing agents, buffers and preservatives.

The compositions can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, can be used.

Compositions can be formulated using one or more physiologically-acceptable carriers comprising excipients and auxiliaries, which facilitate processing of the microorganisms (or, e.g., protein components therefrom) into preparations that can be used pharmaceutically. Formulation may be modified depending upon the route of administration chosen. Compositions described herein may be manufactured, for example, by means of conventional mixing, dissolving, granulating, vitrification, spray-drying, lyophilizing, dragee-making, levigating, encapsulating, entrapping, emulsifying or compression processes.

In some embodiments, the composition is manufactured in a dry form, for example, by spray-drying or lyophilization. In some embodiments, the formulation is prepared as a liquid capsule to maintain the liquid form of the microbes or protein components therefrom.

Compositions provided herein can be stored at any suitable temperature. The formulation can be stored in cold storage, for example, at a temperature of about −80° C., about −20° C., about −4° C., or about 4° C. The storage temperature can be, for example, about 0° C., about 1° C., about 2° C., about 3° C., about 4° C., about 5° C., about 6° C., about 7° C., about 8° C., about 9° C., about 10° C., about 12° C., about 14° C., about 16° C., about 20° C., about 22° C., or about 25° C. In some embodiments, the storage temperature is between about 2° C. to about 8° C. Storage of microbial compositions at low temperatures, for example from about 2° C. to about 8° C., can keep the microbes alive and increase the efficiency of the composition, for example, when present in a liquid or gel formulation. Storage at freezing temperature, below 0° C., with a cryoprotectant can further extend stability.

The pH of the composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. If the pH is outside the range desired by the formulator, the pH can be adjusted by using sufficient pharmaceutically-acceptable acids and bases. In some embodiments, the pH of the composition is between about 4 and about 6.

Compositions containing microbes described herein and/or protein components therefrom can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Microbial compositions can also be administered to lessen a likelihood of developing, contracting, or worsening a condition. Amounts effective for this use can vary based on the severity and course of the disease or condition, previous therapy, the subject's health status, weight, and response to the drugs, and the judgment of the treating physician.

In some embodiments, combining one or more microbes or protein components therefrom in a composition can provide a synergistic effect when administered to the individual. For example, administration of a first microbe may be beneficial to a subject and administration of a second microbe may be beneficial to a subject but when the two microbes are administered together to a subject, the benefit is greater than the either benefit alone.

Different types of microbes or protein components in a composition can be present in the same amount or in different amounts. For example, the ratio of two microbes in a composition can be about 1:1, 1:2, 1:5, 1:10, 1:25, 1:50, 1:100, 1:1000, 1:10,000, or 1:100,000.

In some embodiments, a composition comprises at least one primary fermenter (e.g., a microbe that generates a substrate such as lactate or acetate) and at least one secondary fermenter (e.g., a microbe that utilizes the substrate produced by the primary fermenter to produce a secondary product such as butyrate). In some embodiments, a composition comprises at least one primary fermenter, at least one secondary fermenter, and at least one prebiotic (e.g., to serve as an energy source for the primary and/or secondary fermenter).

Microbes may be produced in any suitable medium for growth, some non-limiting examples include: RCM, GYT veg, BHI, PYGveg, nutrient media, minimal media, selective media, differential media, and transport media. The growth medium can comprise a trace mineral.

The growth medium can comprise a salt. The growth medium can comprise a vitamin. The growth medium can comprise a buffer. The pH of a growth medium can be, for example, about 7. The pH of a growth medium can be, for example, about 3, about, 4, about, 5, about 6, about 7, or about 8. The growth medium can improve the maximum density a microbial strain can grow to. The growth medium can allow for higher strain concentrations. The growth medium can buffer acid production by a microbial strain, which can minimize the inhibitory effect of, for example, very low pH.

In some embodiments, the media used for microbial culture is a vegetable-based media that is free of any animal or dairy based ingredients or derivatives. In another embodiment, the media is a meat-free media that is free of any animal-derived components. In an embodiment, the media is a culture medium having a pH of at least 6 and at most 8. In an embodiment, culture medium comprises one or more of a sugar, a yeast extract, a plant-derived peptone, plant-derived hydrolysate, cysteine, magnesium, calcium, potassium, and a vitamin; and lacks any animal, meat, or dairy based ingredients or derivatives. In an embodiment, the microbes are cultured under anaerobic conditions. In an embodiment, the microbes are lyophilized under anaerobic conditions.

Methods of Treating a Subject

The disclosure provides methods for treating a subject or managing a health condition. Altering the composition of a microbiome in a subject can have desired health consequences. Compositions of the disclosure can be administered as a therapeutic, nutritional/dietary supplement, and/or a cosmetic for treating a health condition. Treatments designed to alter the host microbiome(s) can result in a reduction of patient symptoms, prevention of disease, and or treatment of the disease or health condition. For example, modification of the gut microbiome can reduce the risk for health conditions such as gut and gut related disorders.

Compositions disclosed herein can be used for the dietary management of a metabolic or gut disorder. Compositions disclosed herein can be used for the dietary management of irritable bowel syndrome, inflammatory bowel disease, ulcerative colitis, diarrhea, constipation, leaky intestine, and/or Crohn's disease. Compositions disclosed herein can be used for the dietary management of visceral motor reflex in the colon of subject. Compositions disclosed herein can be used for the dietary management of pain in response to colorectal distension in a subject.

In practicing the methods of treatment or use provided herein, therapeutically-effective amounts of the microbial compositions described herein are administered to a subject having a disease or condition to be treated. In some embodiments, the subject is a mammal such as a human A therapeutically-effective amount can vary widely depending on the severity of the disease, the age and relative health of the subject, potency of the formulation, and other factors. Subjects can be, for example, humans, elderly adults, adults, adolescents, pre-adolescents, children, toddlers, infants, or neonates. A subject can be a patient. A subject can be an individual enrolled in a clinical study. A subject can be a laboratory animal, for example, a mammal, or a rodent.

In certain embodiments, the disclosure provides methods for the restoration of a microbial habitat of a subject to a healthy state. The method can comprise microbiome correction and/or adjustment including for example, replenishing native microbes, removing pathogenic microbes, administering prebiotics, and growth factors necessary for microbiome survival. In some embodiments, the method also comprises administering antimicrobial agents such as antibiotics.

The present disclosure provides methods for generalized-treatment recommendation for a subject as well as methods for subject-specific treatment recommendation. Such methods may be based on a microbiome profile of the subject. Methods for treatments can comprise one of the following steps: determining a first ratio of a level of a subject-specific microbiome profile to a level of a second microbiome profile in a biological sample obtained from at least one subject, detecting a presence or absence of a disease in the subject based upon the determining, and recommending to the subject at least one generalized or subject-specific treatment to ameliorate disease symptoms.

Health conditions that can be treated using the formulations described herein include, but are not limited to, irritable bowel syndrome, inflammatory bowel disease, ulcerative colitis, diarrhea, constipation, leaky intestine, and/or Crohn's disease. The present disclosure can provide for a diagnostic assay of at least one microbiome that includes a report that gives guidance on health status or treatment modalities for the health conditions described herein. The present disclosure can also provide therapeutic and/or cosmetic formulations for treatment of health conditions described herein.

Inflammatory bowel disease (IBD) can involve chronic inflammation of all or part of the digestive tract. IBD can lead to ulcerative colitis and/or Crohn's disease. IBD can be painful and debilitating, and sometimes leads to life-threatening complications.

The formulations described herein can be useful in the treatment and/or amelioration or management of specific symptoms. In embodiments, the formulations are used to reduce visceral motor reflex in the colon of subject. In further embodiments, the formulations described herein are used to reduce pain in response to colorectal distension in a subject. In particular embodiments, the visceral motor reflex or colorectal distension is caused by one or more of irritable bowel syndrome, inflammatory bowel disease, ulcerative colitis, diarrhea, constipation, leaky intestine, and Crohn's disease

In some embodiments, the prebiotic and probiotic consortia are chosen to create an entirely self-sufficient system that does not require any external input. For example, a subject with irritable bowel syndrome, inflammatory bowel disease, ulcerative colitis, diarrhea, constipation, leaky intestine, and/or Crohn's disease can be treated with a formulation of the disclosure which includes a prebiotic and possibly other agents. In this manner, the prebiotic and probiotic form a self-sufficient system, wherein the probiotic metabolizes the prebiotic dietary and triggers downstream signaling for controlling and/or ameliorating irritable bowel syndrome, inflammatory bowel disease, ulcerative colitis, diarrhea, constipation, leaky intestine, and/or Crohn's disease in the subject.

Also provided are methods to generate probiotics against a subject's microbiome composition. The microbiome composition can have an effect on the subject's disease status and clinical treatment response. Compositions of the disclosure can be tailored to suit the microbiome composition of a subject for effective treatment of symptoms associated with a health condition. For example, therapeutic formulations for obese individuals can differ from therapeutic formulations for non-obese individuals for the treatment of a specific disorder based on differences in their microbiota.

A formulation can be administered by a suitable method for delivery to any part of the gastrointestinal tract of a subject including oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract.

In some embodiments, administration of a formulation occurs orally, for example, through a capsule, pill, powder, tablet, gel, or liquid, designed to release the composition in the gastrointestinal tract. In some embodiments, administration of a formulation occurs by injection, for example, for a formulation comprising butyrate, propionate, acetate, and short-chain fatty acids. In some embodiments, the administration of a formulation occurs by application to the skin, for example, cream, liquid, or patch. In some embodiments, administration of a formulation occurs by a suppository and/or by enema. In some embodiments, a combination of administration routes is utilized.

In some embodiments, a formulation is administered before, during, and/or after treatment with an antimicrobial agent such as an antibiotic. For example, the formulation can be administered at least about 1 hour, 2 hours, 5 hours, 12 hours, 1 day, 3 days, 1 week, 2 weeks, 1 month, 6 months, or 1 year before and/or after treatment with an antibiotic. The formulation can be administered at most 1 hour, 2 hours, 5 hours, 12 hours, 1 day, 3 days, 1 week, 2 weeks, 1 month, 6 months, or 1 year before and/or after treatment with an antibiotic.

In some embodiments, the formulation is administered after treatment with an antibiotic. For example, the formulation can be administered after the entire antibiotic regimen or course is complete.

In some embodiments, a formulation is administered before, during, and/or after food intake by a subject. In some embodiments, the formulation is administered with food intake by the subject. In some embodiments, the formulation is administered with (e.g., simultaneously) with food intake.

In some embodiments, the formulation is administered before food intake by a subject. In some embodiments, the formulation is more effective or potent at treating a microbial condition when administered before food intake. For example, the formulation can be administered about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at least about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at most about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject.

In some embodiments, the formulation is administered after food intake by the subject. In some embodiments, the formulation is more effective or potent at treating a microbial condition when administered after food intake. For example, the formulation can be administered at least about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject. For example, the formulation can be administered at most about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject.

Multiple therapeutic agents can be administered in any order or simultaneously. If simultaneously, the multiple therapeutic agents can be provided in a single, unified form, or in multiple forms, for example, as multiple separate pills. The composition can be packed together or separately, in a single package or in a plurality of packages. One or all of the therapeutic agents can be given in multiple doses. If not simultaneous, the timing between the multiple doses may vary to as much as about a month.

Compositions described herein can be administered before, during, or after the occurrence of a disease or condition, and the timing of administering the composition can vary. For example, the microbial composition can be used as a prophylactic and can be administered continuously to subjects with a propensity to conditions or diseases in order to lessen a likelihood of the occurrence of the disease or condition. The microbial compositions can be administered to a subject during or as soon as possible after the onset of the symptoms. The administration of the microbial compositions can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A composition can be administered as soon as is practicable after the onset of a disease or condition is detected or suspected, and for a length of time necessary for the treatment of the disease, such as, for example, from about 1 month to about 3 months. The length of treatment can vary for each subject.

Compositions described herein may be administered in combination with another therapy, for example, immunotherapy, chemotherapy, radiotherapy, anti-inflammatory agents, anti-viral agents, anti-microbial agents, and anti-fungal agents.

A composition of the disclosure can be administered in combination with another therapeutic agent for a metabolic or gut disorder. In some embodiments, a composition of the disclosure can be administered in combination with another therapeutic agent for irritable bowel syndrome, inflammatory bowel disease, ulcerative colitis, diarrhea, constipation, leaky intestine, and Crohn's disease. In some embodiments, a composition of the disclosure can be administered in combination with a therapeutic agent for irritable bowel syndrome. In some embodiments, the other therapeutic agent can serve as an adjuvant in modulating, potentiating, or boosting the effect of a composition of the disclosure in the subject.

Compositions described herein may be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

Dosing

The appropriate quantity of a therapeutic or cosmetic composition to be administered, the number of treatments, and unit dose can vary according to a subject and/or the disease state of the subject.

Compositions described herein can be in unit dosage forms suitable for single administration of precise dosages. In unit dosage form, the formulation can be divided into unit doses containing appropriate quantities of one or more microbial compositions (e.g., comprising one or more microbes and/or protein components of the disclosure). The unit dosage can be in the form of a package containing discrete quantities of the formulation. Non-limiting examples are liquids in vials or ampoules. Aqueous suspension compositions can be packaged in single-dose non-reclosable containers. The composition can be in a multi-dose format. Multiple-dose reclosable containers can be used, for example, in combination with a preservative. Formulations for parenteral injection can be presented in unit dosage form, for example, in ampoules, or in multi-dose containers with a preservative.

The dosage can be in the form of a solid, semi-solid, or liquid composition. Non-limiting examples of dosage forms suitable for use include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, dietary supplement, and any combination thereof.

A microbe can be present in any suitable concentration in a composition. The concentration of a microbe can be for example, from about 10¹ to about 10¹⁸ colony forming units (CFU). The concentration of a microbe can be, for example, at least 10¹, at least 10², at least 10³, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, or at least 10¹⁸ CFU. The concentration of a microbe can be, for example, at most 10¹, at most 10², at most 10³, at most 10⁴, at most 10⁵, at most 10⁶, at most 10⁷, at most 10⁸, at most 10⁹, at most 10¹⁰, at most 10¹¹, at most 10¹², at most 10¹³, at most 10¹⁴, at most 10¹⁵, at most 10¹⁶, at most 10¹⁷, or at most 10¹⁸ CFU.

In some embodiments, the concentration of a microbe is from about 10⁸ CFU to about 10⁹ CFU. In some embodiments, the concentration of a microbe is about 10⁸ CFU. In some embodiments, the concentration of a microbe is about 10⁹ CFU.

Compositions as described herein may be formulated with any suitable therapeutically-effective concentration of prebiotic. For example, the therapeutically-effective concentration of a prebiotic can be at least about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be at most about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. In some embodiments, the concentration of a prebiotic in a composition is about 70 mg/ml. In some embodiments, the prebiotic is inulin.

Compositions as described herein may be administered, for example, 1, 2, 3, 4, 5, or more times daily. Compositions may be administered, for example, daily, every other day, three times a week, twice a week, once a week, or at other appropriate intervals for treatment of the condition.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1a—Effect of Composition on Visceral Hypersensitivity in a Neonatal Mouse Model

Visceral hypersensitivity was induced by infusing 0.5% acetic acid (AA) or equal volume of saline solution as control directly into the colorectum of neonatal mice (C57BL/6) at postnatal days 9 or 10. Each group contained 8 male mice. The pups were weaned at 3 weeks of age and allowed to grow up normally. At 12-24 weeks of age, the mice were administered synbiotic formulations or negative control via daily oral gavage for 14 days (Table 1). Each mouse received about 30 mg of lyophilized powder resuspended in 200 uL anaerobic PBS a day (about 1 g/kg). Then visceral hypersensitivity was tested by measuring visceral motor reflection (VMR) via electromyography recordings of the abdominal muscles in response to colorectal distension (CRD). All mice were returned to the cages for 7 days without dosing. On the seventh day of the washout period (Day 22), mice were subject to a second measure of VMR to CRD and sacrificed. Stools were collected before dosing (Day 1), after the last day of dosing before the first CRD (Day 15), and after the 7 days washout before the second CRD (Day 22).

TABLE 1

| | Blind | | Colorectal | # of | Synbiotic | Test | Sac |
|---|---|---|---|---|---|---|---|
| Group | name | Tested function | infusion | mice | Days | Days | Day |
| Saline/ | Control | No condition control - | Saline | 8 | SYN5 | 15, 22 | 22 |
| control | | mice not sensitized | | | 1-14 | | |
| AA/ | C | Assay control - mice | 0.5% AA | 8 | SYN5 | 15, 22 | 22 |
| control | | sensitized w/o treatment | | | 1-14 | | |
| AA/ | A | Butyrate + | 0.5% AA | 8 | SYN1 | 15, 22 | 22 |
| WB- | | H2 removal + | | | 1-14 | | |
| SYN1 | | mucin association | | | | | |
| AA/ | D | Butyrate + | 0.5% AA | 8 | SYN2 | 15, 22 | 22 |
| WB- | | H2 removal | | | 1-14 | | |
| SYN2 | | | | | | | |
| AA/ | E | Butyrate only | 0.5% AA | 8 | SYN3 | 15, 22 | 22 |
| WB- | | | | | 1-14 | | |
| SYN3 | | | | | | | |
| AA/ | B | No metabolism of any | 0.5% AA | 8 | SYN4 | 15, 22 | 22 |
| WB- | | kind | | | 1-14 | | |
| SYN4 | | | | | | | |

WBF-SYN1 contains Bifidobacterium adolescentis, Ruminococcus faecis, Blautia producta, Anaerostipes caccae, Coprococcus comes, and inulin.

WBF-SYN2 contains Blautia producta, Anaerostipes caccae, Coprococcus comes and inulin.

WBF-SYN3 contains Anaerostipes caccae, Coprococcus comes and inulin.

WBF-SYN4 contains pasteurized five-strain group as microbes of WBF-SYN1.

The control (WBF-30) contains sucrose, polyvinylpyrrolidone, and inulin.

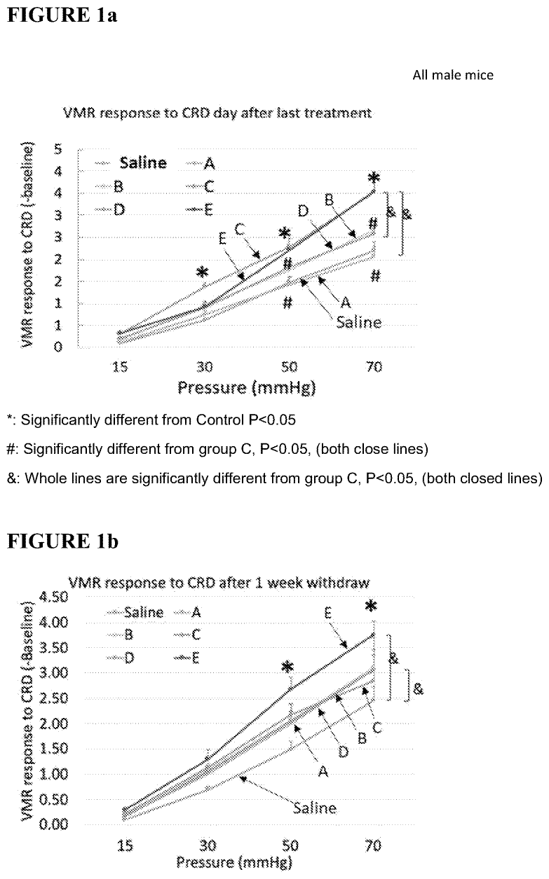

As can be seen from FIG. 1a, both WBF-SYN1 (Group A) displayed marked reduction in VMR response after treatment as compared to the assay control (Group C). The three-strain subset WBF-SYN2 (Group D) and pasteurized five-strain group (WBF-Syn4, Group B) also displayed significantly reduced VMR response compared to the negative control (Group C). The two-strain subset (WBF-Syn3; Group E) was not different from the negative control (Group C).

After withdrawal from treatment for 1 week, VMR responses to CRD returned to the level of negative control (Group C) for the five-, three-strain and pasteurized groups (Groups A, D, B) (FIG. 1b).

### Example 1b—Effect of Compositions on Colonic Hyperalgesia in a Mouse Model of IBS

The IBS model was induced by mild neonatal colorectal irritation in C57B/6 mice. Twenty microliters of 0.5% acetic acid (AA) or saline was infused in the colorectum of mice during postnatal day 9-12. Each group contained 8 mice of mixed gender. The pups were weaned at 3 weeks of age and allowed to grow up normally. At 8-12 weeks of age, the mice (one saline and five acetic acid (IBS) groups) were administered WBF-14, WBF-29, or control solution (see table 2 below) by daily gavage for 2 weeks. Then, colonic pain sensitivity of the mice was tested using the visceral motor reflex (VMR) response (measured by EMG recordings of abdominal muscle) to colorectal distension (CRD). Two groups of mice treated with WBF-14, WBF-29 were then sacrificed after the VMR test. The remaining four groups of IBS mice were kept alive for VMR to CRD days later to assess the persistence of the effect, if any, after withdrawal of WBF-14, WBF-29, and sacrificed afterward.

TABLE 2

| | Colorectal | | Treatment | |
|---|---|---|---|---|
| | infusion | # of | (adult) for | Test/ |
| Group | (neonatal) | animals | 2 weeks | Sacrifice |
| Saline/ | Saline | 8 | Control | Day 15, 21/ |
| control | | | | Day 21 |
| AA/Control | 0.5% acetic acid | 8 | Control | Day 15, 21/ |
| | | | | Day 21 |
| AA/WBF-14 | 0.5% acetic acid | 8 | WBF-14 | Day 15/Day 15 |
| AA/WBF-29 | 0.5% acetic acid | 8 | WBF-29 | Day 15/Day 15 |
| AA/WBF-14/ | 0.5% acetic acid | 8 | WBF-14 | Day 15, 21/ |
| withdrawal | | | | Day 21 |
| AA/WBF-29/ | 0.5% acetic acid | 8 | WBF-29 | Day 15, 21/ |
| withdrawal | | | | Day 21 |

WBF-14 contains Bifidobacterium infantis, Akkermansia muciniphila, Clostridium butyricum, and inulin.

WBF-29 contains Bifidobacterium adolescentis, Ruminococcus faecis, Blautia producta, Anaerostipes caccae, and Coprococcus comes, and inulin.

The control (WBF-30) contains sucrose, polyvinylpyrrolidone, and inulin.

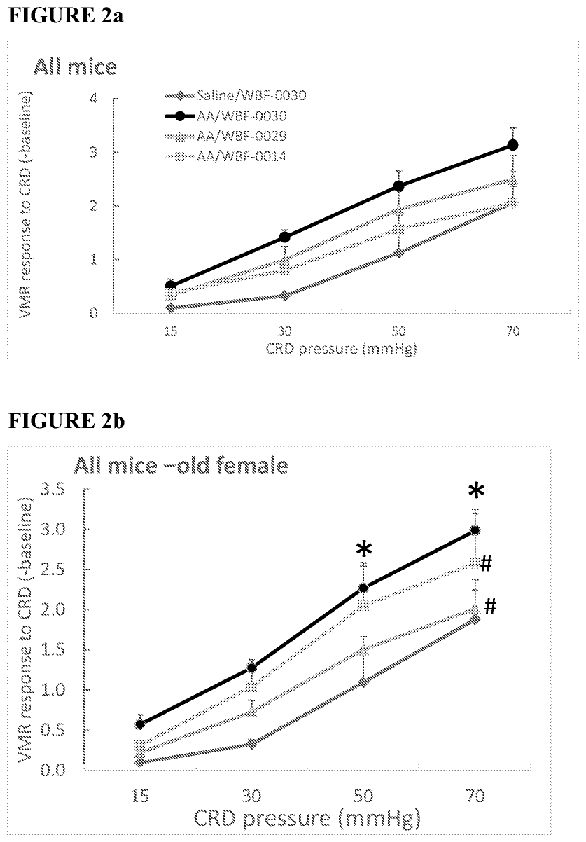

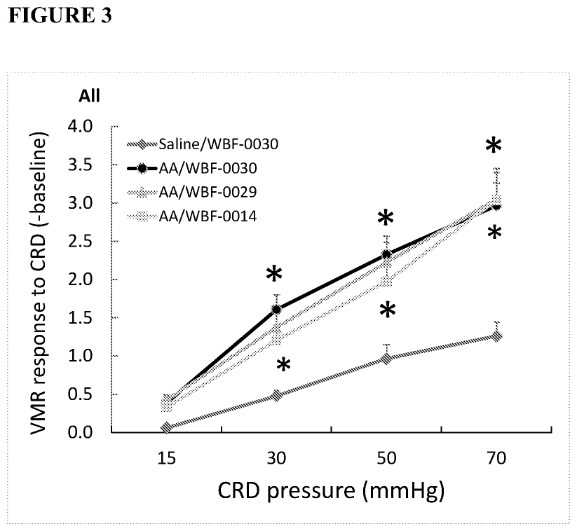

As can be seen from FIG. 2a, both WBF-14 (p=0.01) and WBF-29 (p=0.059) displayed marked reduction in VMR response after treatment as compared to WBF-30 IBS animals (control). When the results were limited to female mice (FIG. 2b), the results were statistically significant across both WBF-14 (p=0.006) and WBF-29 (p=0.002) as compared to the WBF-30 control). After withdrawal from treatment for 1 week, VMR responses returned to normal (FIG. 3).

### Example 2—Effect of Compositions on EMG Response

The IBS model was induced by treatment with acetic acid as in Example 1. Test animals were treated with WBF-12 (test) or WBF-13 (control). EMG recordings for abdominal muscle were recorded in response to CRD at various pressures.

WBF-12 contains Bifidobacterium infantis, Akkermansia muciniphila, Clostridium butyricum, Eubacterium hallii, Clostridium beijerinckii, and inulin.

WBF-13 (control) contains sucrose, trehalose, inulin, polyvinylpyrrolidone, and skim milk.

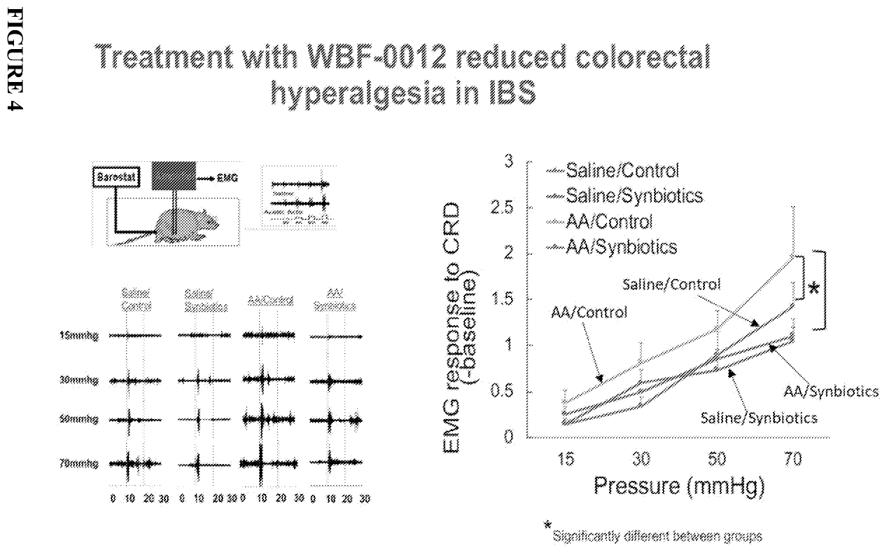

As can be seen in FIG. 4, the treatment with WBF-12 significantly reduced (p<0.05) the EMG response in the treated animals. AA denotes treatment with acetic acid. “Synbiotic” refers to treatment with WBF-12. “Control” refers to WBF-13.

### Example 3—Effect of Compositions on Anxiety Like Behavior

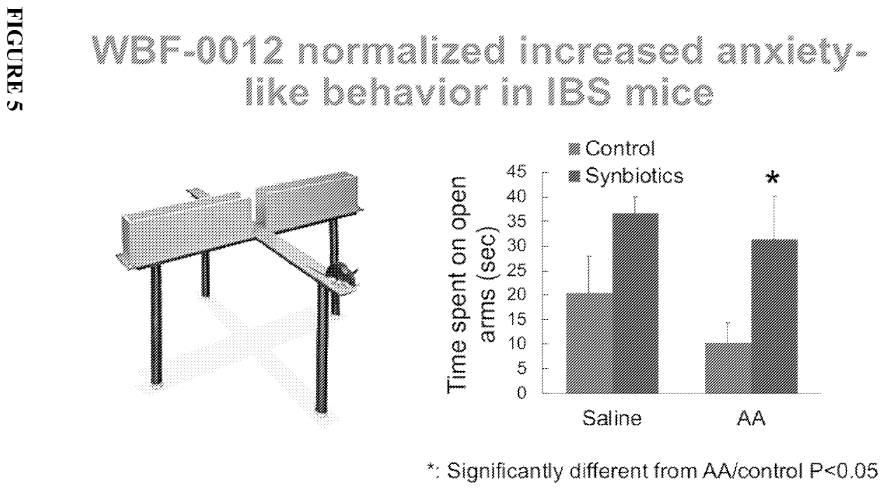

The IBS model was induced by treatment with acetic acid as in Example 1. Test animals were treated with WBF-12 (test) or WBF-13 (control) (as above). Mice were individually placed in an elevated plus maze (see FIG. 5) and the amount of time spent on the open arms was measured. As can be seen in FIG. 5, mice treated with WBF-12 spent statistically significant more time on the open arms of the test table. These results indicate that WBF-12 reduced anxiety as compared to control animals.

### Example 4—Effect of Compositions on Sensory Neurons from Dorsal Root Ganglia

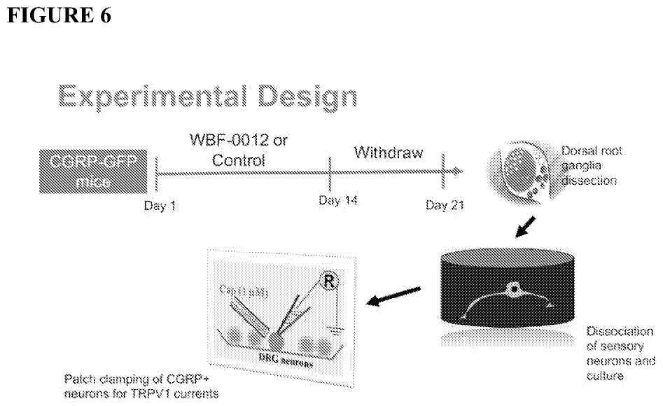

The IBS model was induced by treatment with acetic acid as in Example 1. Test animals were treated with WBF-12 (test) or WBF-13 (control) (as above). The experimental design was as represented in FIG. 6. After 14 days of treatment a first group was sacrificed and dorsal root ganglia was isolated. CGRP+ neurons were patch clamped and tested for TRPV1 (vaniloid receptors) currents. After a further week of withdrawal from treatment, the second group was sacrificed and dorsal root ganglia was isolated. CGRP+ neurons were patch clamped and tested for TRPV1 (vaniloid receptors) currents.

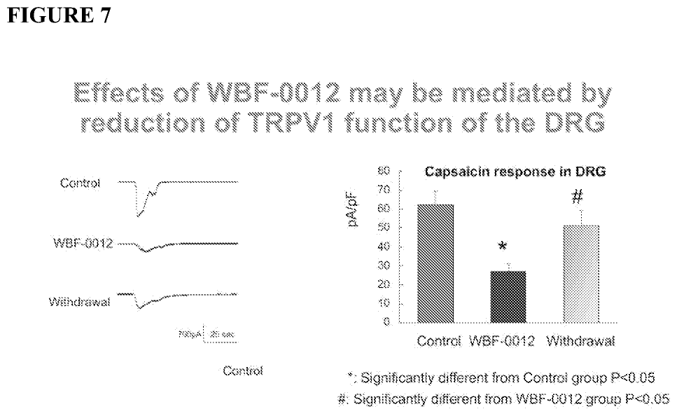

As can be seen in FIG. 7, mice treated with WBF-12 displayed statistically significantly reduced TRPV1 function as compared to control animals. Further the withdrawal of treatment for one week resulted in recovery of TRPV1 function. These results indicate that WBF-12 reduced anxiety as compared to control animals.

### Example 5—Effect of Compositions on Expression of Neuronal Nitric Oxide Synthase (nNOS) and HuC (ELAV-Like Protein 3)/HuD (ELAV-Like Protein 4) Expression

The IBS model was induced by treatment with acetic acid as in Example 1. Test animals were treated for two weeks with WBF-12 (test) or WBF-13 (control) (as above). The mice were sacrificed and the longitudinal smooth muscle-myenteric plexus (LMMP) was removed and stained for nNOS (rabbit anti-nNOS 1:500) and Hu C/D (human anti-Hu C/D 1:2000).

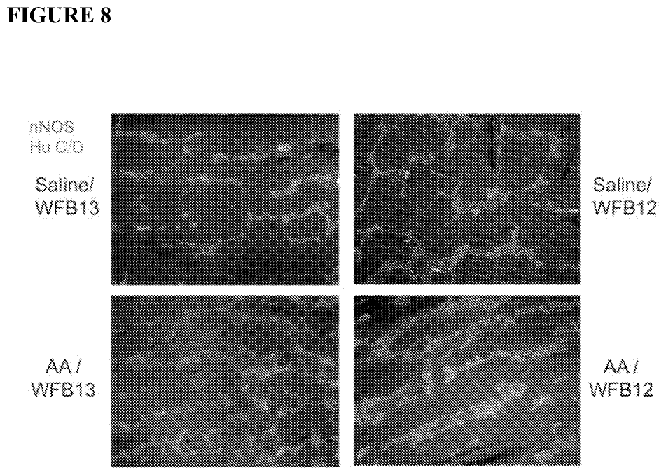

Representative images of the stainings are presented in FIG. 8, with nNOS appearing in red and Hu C/D is in green.

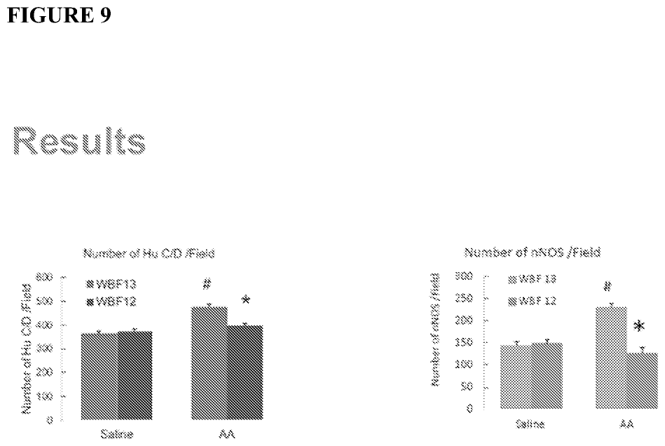

As can be seen in FIG. 9, mice treated with WBF-12 displayed statistically significantly reduced Hu C/D and nNOS staining as compared to control animals.

What is claimed is:

1. A composition comprising one or more microbes selected from the group consisting of Akkermansia sp., Anaerostipes sp., Bacteroides sp., Bifidobacterium sp., Blautia sp., Clostridium sp., Collinsella sp., Coprococcus sp., Eubacterium sp., and Ruminococcus sp.

2. The composition of claim 1, wherein the composition comprises microbes from 2 or more, 3 or more, four or more, five or more, six or more, seven or more, eight or more, nine or more, or all ten of the group consisting of Akkermansia sp., Anaerostipes sp., Bacteroides sp., Bifidobacterium sp., Blautia sp., Clostridium sp., Collinsella sp., Coprococcus sp., Eubacterium sp., and Ruminococcus sp.

3. The composition of claim 1, wherein the microbes are selected from 2 or more, 3 or more, four or more, or all five of the group consisting of Bifidobacterium sp., Ruminococcus sp., Blautia sp., Anaerostipes sp., and Coprococcus sp.

4. The composition of claim 1, wherein the microbes are selected from 2 or more, 3 or more, or all four of the group consisting of Bifidobacterium sp., Akkermansia sp., Clostridium sp., and Eubacterium sp.

5. The composition of claim 1, wherein the microbes are selected from 2 or more, 3 or more, four or more, five or more, six or more, or all seven of the group consisting of Eubacterium sp., Clostridium sp., Bifidobacterium sp., Collinsella sp., Bacteroides sp., Blautia sp., and Bacteroides sp.

6. A composition comprising one or more microbes having a 16S rRNA sequence comprising at least 95% identity to the full length of a 16S rRNA sequence of a microbe selected from the group consisting of Akkermansia muciniphila ATCC BAA-835, Anaerostipes caccae DSM 14662, Bacteroides finegoldii DSM 7565, Bacteroides ovatus ATCC 8483, Bacteroides stercoris ATCC 43183, Bifidobacterium adolescentis ATCC 15703, Bifidobacterium infantis ATCC 15697, Bifidobacterium faecale JCM 19861, Bifidobacterium longum ATCC 15697, Blautia hydrogenotrophica DSM 10507, Blautia producta ATCC 27340, Clostridium butyricum DSM 10702, Clostridium beijerinckii NCIMB 8052, Clostridium innocuum ATCC 14501, Clostridium sporogenes DSM 795, Collinsella aerofaciens ATCC 25986, Coprococcus comes ATCC 27758, Eubacterium hallii DSM 3353, Eubacterium limosum ATCC 5486, and Ruminococcus faecis JCM 15917.

7. The composition of claim 6, wherein the composition comprises microbes having 16S rRNA sequence comprising at least 95% identity to the full length of a 16S rRNA sequence of a microbe from 2 or more, 3 or more, four or more, five or more, six or more, seven or more, eight or more, nine or more, ten or more, eleven or more, or all twelve of the group consisting of Eubacterium limosum ATCC 5486, Clostridium innocuum ATCC 14501, Bifidobacterium faecale JCM 19861, Collinsella aerofaciens ATCC 25986, Bacteroides stercoris ATCC 43183, Bifidobacterium adolescentis ATCC 15703, Bifidobacterium infantis ATCC 15697, Bifidobacterium longum ATCC 15697, Clostridium sporogenes DSM 795, Blautia hydrogenotrophica DSM 10507, Bacteroides ovatus ATCC 8483, and Bacteroides finegoldii DSM 7565.

8. The composition of claim 6, wherein the composition comprises microbes having 16S rRNA sequence comprising at least 95% identity to the full length of a 16S rRNA sequence of a microbe from 2 or more, 3 or more, four or more, or all five of the group consisting of Bifidobacterium infantis ATCC 15697, Akkermansia muciniphila ATCC BAA-835, Clostridium butyricum DSM 10702, Eubacterium hallii DSM 3353, Clostridium beijerinckii NCIMB 8052.

9. The composition of claim 6, wherein the composition comprises microbes having 16S rRNA sequence comprising at least 95% identity to the full length of a 16S rRNA sequence of a microbe from 2 or more, 3 or more, four or more, or all five of the group consisting of Bifidobacterium adolescentis ATCC 15703, Ruminococcus faecis JCM 15917, Blautia producta ATCC 27340, Anaerostipes caccae DSM 14662, and Coprococcus comes ATCC 27758.

10. A composition comprising one or more microbes selected from the group consisting of Akkermansia muciniphila, Anaerostipes caccae, Bacteroides finegoldii, Bacteroides ovatus, Bacteroides stercoris, Bifidobacterium adolescentis, Bifidobacterium infantis, Bifidobacterium faecale, Bifidobacterium longum, Blautia hydrogenotrophica, Blautia producta, Clostridium butyricum, Clostridium beijerinckii, Clostridium innocuum, Clostridium sporogenes, Collinsella aerofaciens, Coprococcus comes, Eubacterium hallii, Eubacterium limosum, and Ruminococcus faecis.

11. The composition of claim 10, wherein the composition comprises microbes from 2 or more, 3 or more, four or more, or all five of the group consisting of Bifidobacterium infantis, Akkermansia muciniphila, Clostridium butyricum, Eubacterium hallii, Clostridium beijerinckii.

12. The composition of claim 10, wherein the composition comprises microbes from 2 or more, 3 or more, four or more, five or more, six or more, seven or more, eight or more, nine or more, ten or more, eleven or more, or all twelve of the group consisting of Eubacterium limosum, Clostridium innocuum, Bifidobacterium faecale, Collinsella aerofaciens, Bacteroides stercoris, Bifidobacterium adolescentis, Bifidobacterium infantis, Bifidobacterium longum, Clostridium sporogenes, Blautia hydrogenotrophica, Bacteroides ovatus, Bacteroides finegoldii.

13. The composition of claim 10, wherein the composition comprises microbes from 2 or more, 3 or more, four or more, or all five of the group consisting of Bifidobacterium adolescentis, Ruminococcus faecis, Blautia producta, Anaerostipes caccae, and Coprococcus comes.

14. The composition of any one of claims 1-13, wherein the composition wherein the composition comprises one or more additional microbes strains having a 16S rRNA sequence comprising at least 95% identity to the full length of a 16S rRNA sequence of a microbe selected from the group consisting of Akkermansia muciniphila, Anaerostipes caccae, Bacteroides finegoldii, Bacteroides ovatus, Bacteroides stercoris, Eubactrium hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium butyricum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof.

15. The composition of any one of claims 1-13, wherein the composition reduces visceral motor reflex in the colon of subject treated with the composition.

16. The composition of any one of claims 1-13, wherein the composition reduces pain in response to colorectal distension in a subject treated with the composition.

17. The composition of any one of claims 15 and 16, wherein the subject suffers from irritable bowel syndrome, inflammatory bowel disease, ulcerative colitis, diarrhea, constipation, leaky intestine, and/or Crohn's disease

18. The composition of any one of claims 15 and 16, wherein the subject is a mammal.

19. The composition of claim 18, wherein the mammal is a human.

20. The composition of any one of claims 1-13, wherein the composition further comprises an enteric coating.

21. The composition of any one of claims 1-13, wherein the composition further comprises an effective amount of a preservative.

22. The composition of any one of claims 1-13, wherein the composition further comprises a prebiotic.

23. The composition of claim 22, wherein the prebiotic is selected from the group consisting of inulin, green banana, reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof.

24. The composition of any one of claims 1-13, wherein at least one of the one or more microbes is lyophilized.

25. The composition of any one of claims 1-13, wherein at least one of the one or more microbes is viable.

26. The composition of any one of claims 1-13, wherein at least one of the one or more microbes is non-viable.

27. The composition of any one of claims 1-13, wherein at least one of the one or more microbes has been pasteurized.

28. A method of treating a subject, the method comprising administering to a subject the composition of any one of claims 1-13.

29. A method of reducing visceral motor reflex in the colon of a subject, the method comprising administering to a subject the composition of any one of claims 1-13.

30. A method of reducing pain in response to colorectal distension in a subject, the method comprising administering to a subject the composition of any one of claims 1-13.

31. The method according to any one of claims 28-30, wherein the subject suffers from irritable bowel syndrome, inflammatory bowel disease, ulcerative colitis, diarrhea, constipation, leaky intestine, and/or Crohn's disease.

32. The method according to any one of claims 28-30, wherein the subject is a mammal.

33. The method according to claim 32, wherein the mammal is a human.

34. The method according to any one of claims 28-30, wherein the composition further comprises an enteric coating.

35. The method according to any one of claims 28-30, wherein the composition further comprises an effective amount of a preservative.

36. The method according to any one of claims 28-30, wherein the composition further comprises a prebiotic.

37. The method according to claim 36, wherein the prebiotic is selected from the group consisting of inulin, green banana, reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof.

38. The method according to any one of claims 28-30, wherein at least one of the one or more microbes is lyophilized.

39. The method according to any one of claims 28-30, wherein at least one of the one or more microbes is viable.

40. The method according to any one of claims 28-30, wherein at least one of the one or more microbes is non-viable.

41. The method according to any one of claims 28-30, wherein at least one of the one or more microbes has been pasteurized.

42. The composition of any one of claims 1-13, wherein the composition is formulated as a pharmaceutical formulation.

43. The composition of any one of claims 1-13, wherein the composition is formulated as a nutritional supplement.

44. The composition of any one of claims 1-13, wherein the composition is formulated as a dietary supplement.

45. The composition of any one of claims 1-13, wherein the composition is formulated as a medical food.
